(19) United States
(12) Patent Application Publication
(10) Pub. No.: US 2026/0337182 A1
(43) Pub. Date: Oct. 29, 2026

(54) COMPOUNDS AND COMPOSITIONS USEFUL AS INHIBITORS OF IAPs

(71) Applicants: Tract Pharmaceuticals, Inc., West Hartford, CT (US); Trustees of Tufts College, Medford, MA (US)

(72) Inventors: Richard E. Russell, Scituate, MA (US); William W. Bachovchin, Moultonborough, NH (US); Hung-Sen Lai, Andover, MA (US); Wengen Wu, Winchester, MA (US)

(21) Appl. No.: 19/116,478

(22) PCT Filed: Sep. 29, 2023
(86) PCT No.: PCT/US2023/034219

§ 371 Date: Mar. 28, 2025

Related U.S. Application Data
(60) Provisional application No. 63/411,550, filed on Sep. 29, 2022.

(51) Int. Cl. C07D 403/12; A61K 31/4025; A61K 31/407; A61K 31/4192; A61K 31/427; A61P 11/00; C07D 417/04; C07D 487/04; C07D 519/00

(52) U.S. Cl. CPC C07D 403/12; A61K 31/4025; A61K 31/407; A61K 31/4192; A61K 31/427; A61P 11/00; C07D 417/04; C07D 487/04; C07D 519/00

(57) ABSTRACT

The present disclosure provides compounds, pharmaceutically acceptable compositions thereof, and methods of using the same.

### TECHNICAL FIELD OF INVENTION

The present disclosure relates to compounds and methods useful for inhibition of Inhibitors of apoptosis proteins (IAPs). The disclosure also provides pharmaceutically acceptable compositions comprising compounds of the present disclosure and methods of using said compositions in the treatment of various diseases, disorders, and conditions as described herein.

### BACKGROUND OF THE INVENTION

IAPs play an important role in controlling cancer cell survival. IAPs have therefore attracted considerable attention as potential targets in treating diseases, disorders, or conditions associated with IAPs.

### SUMMARY OF THE INVENTION

In some embodiments, the present disclosure provides the recognition that there remains a need to find inhibitors of IAPs useful as therapeutic agents. It has now been found that compounds of the present disclosure, and pharmaceutically acceptable salts and compositions thereof, are effective as inhibitors of IAPs. In some embodiments, the present disclosure provides a compound of formula I:
or a pharmaceutically acceptable salt thereof, wherein: L1 is a first ligand; L2 is a second

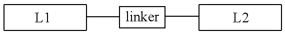

ligand; and linker is a bivalent linker comprising

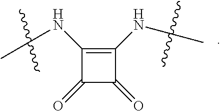 [SMILES: *Nc1c(N*)c(=O)c1=O]

Compounds described herein, and pharmaceutically acceptable compositions thereof, are useful for treating a variety of diseases, disorders, or conditions associated with IAPs. Such diseases, disorders, or conditions include those described herein.

Compounds provided herein are also useful for the study of IAPs in, e.g., biological and pathological phenomena, and the comparative evaluation of new IAP inhibitors.

### BRIEF DESCRIPTION OF THE DRAWING

FIG. 1 depicts treatment of Barrett's esophagus cells with compounds described herein. The concentration of the compound used and percent survival of the cells is provided.

FIG. 2 depicts treatment of esophageal adenocarcinoma stem cells with compounds described herein. The concentration of the compound used and percent survival of the cells is provided.

FIG. 3 depicts treatment of lung adenocarcinoma stem cells with compound I-1. FIG. 3A depicts the % survival of lung adenocarcinoma stem cells at higher concentration of compound I-1. FIG. 3B depicts the % survival of lung adenocarcinoma stem cells at lower concentration of compound I-1. Regardless of concentration, compound I-1 demonstrates comparable lethality to another highly potent cytotoxic treatment.

FIG. 4 depicts treatment of advanced Barrett's esophagus stem cells with compound I-1 in cells isolated from two different patients. FIG. 4A depicts the % survival of Barrett's esophagus stem cells from a first patient at higher concentration of compound I-1. FIG. 4B depicts the % survival of Barrett's esophagus cells from a first patient at lower concentration of compound I-1. FIG. 4C depicts the % survival of Barrett's esophagus cells from a second patient at higher concentration of compound I-1. FIG. 4D depicts the % survival of Barrett's esophagus stem cells from a second patient at lower concentration of compound I-1.

FIG. 5 depicts treatment of Barrett's high grade dysplasia and diffuse gastric cancer with compound I-1. FIG. 5A depicts the % survival of Barrett's high grade dysplasia stem cells at higher concentration of compound I-1. FIG. 5B depicts the % survival of Barrett's high grade dysplasia stem cells at lower concentration of compound I-1. FIG. 5C depicts the % survival of diffuse gastric cancer stem cells at higher concentration of compound I-1. FIG. 5D depicts the % survival of diffuse gastric cancer stem cells at lower concentration of compound I-1.

FIG. 6 depicts treatment of Barrett's low grade dysplasia and esophageal adenocarcinoma with compound I-1. FIG. 6A depicts the % survival of Barrett's low grade dysplasia stem cells at higher concentration of compound I-1. FIG. 6B depicts the % survival of Barrett's low grade dysplasia stem cells at lower concentration of compound I-1. FIG. 6C depicts the % survival of esophageal adenocarcinoma stem cells at higher concentration of compound I-1. FIG. 6D depicts the % survival of esophageal adenocarcinoma cells at lower concentration of compound I-1.

FIG. 7 depicts treatment of various diseases with compounds I-2 and I-5. FIG. 7A depicts % survival of diffused gastric adenocarcinoma with ascites cells with varying concentrations of compounds I-2 and I-5. FIG. 7B depicts % survival of advanced Barret's cells with varying concentrations of compounds I-2 and I-5. FIG. 7C depicts % survival of high-grade serous ovarian cancer cells with varying concentrations of compounds I-2 and I-5. FIG. 7D depicts % survival of gastric cancer with varying concentrations of compounds I-2 and I-5. FIG. 7E depicts % survival taxol resistant ovarian cancer stem cells with varying concentrations of compounds I-2 and I-5.

FIG. 8 depicts treatment of Barrett's low grade dysplasia and high grade dysplasia with compounds I-1; I-2; I-6; and I-3. FIG. 8A depicts the % survival of Barrett's low grade dysplasia stem cells at varying concentrations of I-1; I-2; I-6; and I-3. FIG. 8B depicts the % survival of Barrett's high grade dysplasia stem cells at varying concentrations of I-1; I-2; I-6; and I-3.

FIG. 9 depicts treatment of pancreatic cancer with compounds I-9, I-10; I-3; I-11. FIG. 9 depicts the % survival of pancreatic cancer stem cells at varying concentrations of I-9; I-10; I-3; I-11.

FIG. 10 depicts the limited toxicity of the compounds described herein on healthy liver stem cells.

FIG. 11 depicts the limited toxicity of the compounds described herein on healthy lung stem cells.

### DETAILED DESCRIPTION OF CERTAIN EMBODIMENTS

### 1. General Description of Certain Embodiments of the Invention

In certain embodiments, the present disclosure provides inhibitors IAPs. In some embodiments, such compounds include those of the formulae described herein, or a pharmaceutically acceptable salt thereof, wherein each variable is as defined and described herein. In some embodiments, the present disclosure provides a compound of formula I:

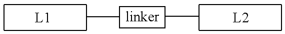

or a pharmaceutically acceptable salt thereof, wherein: L1 is a first ligand; L2 is a second ligand; and linker is a bivalent linker comprising

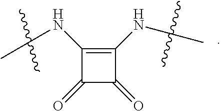 [SMILES: *Nc1c(N*)c(=O)c1=O]

### 2. Compounds and Definitions

Compounds of the present disclosure include those described generally above, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle”, “carbocyclic”, “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated”, as used herein, means that a moiety has one or more units of unsaturation.

As used herein, the term “partially unsaturated”, as used herein, refers to a ring moiety that includes at least one double or triple bond. The term “partially unsaturated”, as used herein, is intended to encompass rings having multiple sites of unsaturation, but is not intended to include aryl or heteroaryl moieties, as herein defined.

The term “lower alkyl”, as used herein, refers to a C₁₋₄ straight or branched alkyl group. Exemplary lower alkyl groups are methyl, ethyl, propyl, isopropyl, butyl, isobutyl, and tert-butyl.

The term “halogen” means F, Cl, Br, or I.

The term “aryl”, as used herein, refers to monocyclic and bicyclic ring systems having a total of five to fourteen ring members, wherein at least one ring in the system is aromatic and wherein each ring in the system contains three to seven ring members. The term “aryl” may be used interchangeably with the term “aryl ring”. In certain embodiments, “aryl” refers to an aromatic ring system which includes, but not limited to, phenyl, biphenyl, naphthyl, anthracyl and the like, which may bear one or more substituents. Also included within the scope of the term “aryl” is a group in which an aromatic ring is fused to one or more non-aromatic rings, such as indanyl, phthalimidyl, naphthimidyl, phenanthridinyl, or tetrahydronaphthyl, and the like.

The term “heteroaryl” as used herein, refers to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” as used herein, refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, pteridinyl, tetrahydroquinolinyl, and tetrahydroisoquinolinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples of heteroaryl rings on compounds of Formula I and subgenera thereof include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, and pyrido[2,3-b]-1,4-oxazin-3(4H)-one. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted.

Additionally, it will be appreciated that, when two groups cyclize to form an optionally substituted heteroaryl ring having at least one nitrogen atom, the nitrogen atom in the ring can be, as valency permits, N or N—R^{†}, as defined infra.

As used herein, the terms “heterocycle”, “heterocyclyl”, and “heterocyclic ring” are used interchangeably and refer to a stable 5- to 7-membered monocyclic or 7- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or +NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, tetrahydroquinolinyl, or tetrahydroisoquinolinyl where the radical or point of attachment is on the heterocyclyl ring. A heterocyclyl group may be mono- or bicyclic.

Additionally, it will be appreciated that, when two groups cyclize to form an optionally substituted heterocyclic ring having at least one nitrogen atom, the nitrogen atom in the ring can be, as valency permits, N or N-Rt, as defined infra.

As described herein, compounds may contain “optionally substituted” moieties. In general, the term “substituted”, whether preceded by the term “optionally” or not, means that one or more hydrogens of the designated moiety of compounds are replaced with a suitable substituent. “Substituted” applies to one or more hydrogens that are either explicit or implicit from the structure (e.g.,

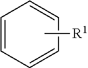

refers to at least

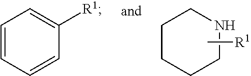

and refers to at least

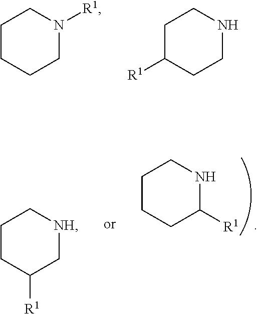

Unless otherwise indicated, an “optionally substituted” group may have a suitable substituent at each substitutable position of the group, and when more than one position in any given structure may be substituted with more than one substituent selected from a specified group, the substituent may be either the same or different at every position.

Suitable monovalent substituents on a substitutable carbon atom of an “optionally substituted” group are independently halogen; —(CH₂)₀₋₄R^{◯}; —(CH₂)₀₋₄OR^{◯}; —O(CH₂)₀₋₄R^{◯}, —O—(CH₂)₀₋₄C(O)OR^{◯}; —(CH₂)₀₋₄CH(OR^{◯})₂; —(CH₂)₀₋₄SR^{◯}; —(CH₂)₀₋₄Ph, which may be substituted with R^{◯}; —(CH₂)₀₋₄O(CH₂)₀₋₁Ph which may be substituted with R^{◯}; —CH═CHPh, which may be substituted with R^{◯}; —(CH₂)₀₋₄O(CH₂)₀₋₁-pyridyl which may be substituted with R^{◯}; —NO₂; —CN; —N₃; —(CH₂)₀₋₄N(R^{◯})₂; —(CH₂)₀₋₄N(R^{◯})C(O)R^{◯}; —N(R^{◯})C(S)R^{◯}; —(CH₂)₀₋₄N(R^{◯})C(O)NR^{◯}₂; —N(R^{◯})C(S)NR^{◯}₂; —(CH₂)₀₋₄N(R^{◯})C(O)OR^{◯}; —N(R^{◯})N(R^{◯})C(O)R^{◯}; —N(R^{◯})N(R^{◯})C(O)NR^{◯}₂; —N(R^{◯})N(R^{◯})C(O)OR^{◯}; —(CH₂)₀₋₄C(O)R^{◯}; —C(S)R^{◯}; —(CH₂)₀₋₄C(O)OR^{◯}; —(CH₂)₀₋₄C(O)SR^{◯}; —(CH₂)₀₋₄C(O)OSiR^{◯}₃; —(CH₂)₀₋₄OC(O)R^{◯}; —OC(O)(CH₂)₀₋₄SR^{◯}, SC(S)SR^{◯}; —(CH₂)₀₋₄SC(O)R^{◯}; —(CH₂)₀₋₄C(O)NR^{◯}₂; —C(S)NR^{◯}₂; —C(S)SR^{◯}; SC(S)SR^{◯}, —(CH₂)₀₋₄OC(O)NR^{◯}₂; —C(O)N(OR^{◯})R^{◯}; —C(O)C(O)R^{◯}; —C(O)CH₂C(O)R^{◯}, —C(NOR^{◯})R^{◯}; —(CH₂)₀₋₄SSR^{◯}, —(CH₂)₀₋₄S(O)₂R^{◯}; —(CH₂)₀₋₄S(O)₂OR^{◯}; —(CH₂)₀₋₄OS(O)₂R^{◯}; —S(O)₂NR^{◯}₂; —(CH₂)₀₋₄S(O)R^{◯}; —N(R^{◯})S(O)₂NR^{◯}₂; —N(R^{◯})S(O)₂R^{◯}; —N(OR^{◯})R^{◯}; —C(NH)NR^{◯}₂; —P(O)₂R^{◯}; —P(O)R^{◯}₂; —OP(O)R^{◯}₂, —OP(O)(OR^{◯})₂; SiR^{◯}₃; —(C₁₋₄ straight or branched alkylene)O—N(R^{◯})₂; or —(C₁₋₄ straight or branched alkylene)C(O)O—N(R^{◯})₂, wherein each R^{◯} may be substituted as defined below and is independently hydrogen, C₁₋₆ aliphatic, —CH₂Ph, —O(CH₂)₀₋₁Ph, —CH₂-(5-to 6 membered heteroaryl ring), or a 5- to 6-membered saturated, partially unsaturated, or aryl ring having 0-4 heteroatoms independently selected from nitrogen, oxygen, or sulfur, or, notwithstanding the definition above, two independent occurrences of R^{◯}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{◯} (or the ring formed by taking two independent occurrences of R^{◯} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{◯} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group of a compound of Formula I, and subgenera thereof, include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5- to 6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5- to 6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3- to 12-membered saturated, partially unsaturated, or aryl mono- or bicyclic ring having 0-4 heteroatoms independently selected from nitrogen, oxygen, or sulfur.

Suitable substituents on the aliphatic group of R are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5- to 6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxyl-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the present disclosure. Unless otherwise stated, all tautomeric forms are within the scope of the disclosure. Additionally, unless otherwise stated, the present disclosure also includes compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this disclosure. Such compounds are useful, for example, as analytical tools, as probes in biological assays, or as therapeutic agents in accordance with the present disclosure. In some embodiments, compounds of this disclosure comprise one or more deuterium atoms.

Combinations of substituents envisioned by this disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

As used herein the term “biological sample” includes, without limitation, cell cultures or extracts thereof; biopsied material obtained from an animal (e.g., mammal) or extracts thereof; and blood, saliva, urine, feces, semen, tears, or other body fluids or extracts thereof; or purified versions thereof. For example, the term “biological sample” refers to any solid or fluid sample obtained from, excreted by or secreted by any living organism, including single-celled micro-organisms (such as bacteria and yeasts) and multicellular organisms (such as plants and animals, for instance a vertebrate or a mammal, and in particular a healthy or apparently healthy human subject or a human patient affected by a condition or disease to be diagnosed or investigated). The biological sample can be in any form, including a solid material such as a tissue, cells, a cell pellet, a cell extract, cell homogenates, or cell fractions; or a biopsy, or a biological fluid. The biological fluid may be obtained from any site (e.g. blood, saliva (or a mouth wash containing buccal cells), tears, plasma, serum, urine, bile, seminal fluid, cerebrospinal fluid, amniotic fluid, peritoneal fluid, and pleural fluid, or cells therefrom, aqueous or vitreous humor, or any bodily secretion), a transudate, an exudate (e.g. fluid obtained from an abscess or any other site of infection or inflammation), or fluid obtained from a joint (e.g. a normal joint or a joint affected by disease such as rheumatoid arthritis, osteoarthritis, gout or septic arthritis). The biological sample can be obtained from any organ or tissue (including a biopsy or autopsy specimen) or may comprise cells (whether primary cells or cultured cells) or medium conditioned by any cell, tissue or organ. Biological samples may also include sections of tissues such as frozen sections taken for histological purposes. Biological samples also include mixtures of biological molecules including proteins, lipids, carbohydrates and nucleic acids generated by partial or complete fractionation of cell or tissue homogenates. Although the sample is preferably taken from a human subject, biological samples may be from any animal, plant, bacteria, virus, yeast, etc. The term animal, as used herein, refers to humans as well as non-human animals, at any stage of development, including, for example, mammals, birds, reptiles, amphibians, fish, worms and single cells. Cell cultures and live tissue samples are considered to be pluralities of animals. In certain exemplary embodiments, the non-human animal is a mammal (e.g., a rodent, a mouse, a rat, a rabbit, a monkey, a dog, a cat, a sheep, cattle, a primate, or a pig). An animal may be a transgenic animal or a human clone. If desired, the biological sample may be subjected to preliminary processing, including preliminary separation techniques.

As used herein, a “disease or disorder associated with IAPs” or, alternatively, “a IAP-mediated disease or disorder” means any disease or other deleterious condition in which an IAP, or a mutant thereof, is known or suspected to play a role. In some embodiments, an IAP is selected from BIRC1/NAIP, BIRC2/cIAP1, BIRC3/cIAP2, BIRC4/XIAP, BIRC5/Survivin, BIRC6/Apollon, BIRC7/ML-IAP and BIRC8/ILP2.

The term “subject”, as used herein, means a mammal and includes human and animal subjects, such as domestic animals (e.g., horses, dogs, cats, etc.). The terms “subject” and “patient” are used interchangeably. In some embodiments, the “patient” or “subject” means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. The amount of compounds described herein that may be combined with the carrier materials to produce a composition in a single dosage form will vary depending upon the host treated, the particular mode of administration, etc.

The expression “unit dosage form” as used herein refers to a physically discrete unit of a provided compound and/or compositions thereof appropriate for the subject to be treated. It will be understood, however, that the total daily usage of the active agent (i.e., compounds and compositions described herein) will be decided by the attending physician within the scope of sound medical judgment. The specific effective dose level for any particular subject (i.e., patient) or organism will depend upon a variety of factors including the disorder being treated and the severity of the disorder; activity of specific active agent employed; specific composition employed; age, body weight, general health, sex and diet of the subject; time of administration, route of administration, and rate of excretion of the specific active agent employed; duration of the treatment; and like factors well known in the medical arts.

The term “parenteral” as used herein includes subcutaneous, intravenous, intramuscular, intra-articular, intra-synovial, intrasternal, intrathecal, intrahepatic, intralesional and intracranial injection or infusion techniques.

As used herein, a “therapeutically effective amount” means an amount of a substance (e.g., a therapeutic agent, composition, and/or formulation) that elicits a desired biological response. In some embodiments, a therapeutically effective amount of a substance is an amount that is sufficient, when administered as part of a dosing regimen to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of a provided compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a “therapeutically effective amount” is at least a minimal amount of a provided compound, or composition containing a provided compound, which is sufficient for treating one or more symptoms of an IAP-mediated disease or disorder.

As used herein, the terms “treatment,” “treat,” and “treating” refer to partially or completely alleviating, inhibiting, delaying onset of, preventing, ameliorating and/or relieving a disorder or condition, or one or more symptoms of the disorder or condition, as described herein. In some embodiments, treatment may be administered after one or more symptoms have developed. In some embodiments, the term “treating” includes preventing or halting the progression of a disease or disorder. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence. Thus, in some embodiments, the term “treating” includes preventing relapse or recurrence of a disease or disorder.

### 3. Description of Exemplary Embodiments

In some embodiments, the present disclosure provides a compound of formula I:

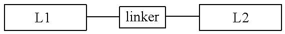

or a pharmaceutically acceptable salt thereof, wherein: L1 is a first ligand; L2 is a second ligand; and linker is a bivalent linker comprising

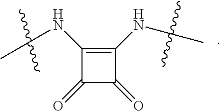 [SMILES: *Nc1c(N*)c(=O)c1=O]

Without wishing to be bound by any particular theory, it is believed that L1 and L2 need to be positioned at a certain distance relative to each other to achieve optimum biological activity. In some embodiments, L1 and L2 need to be positioned at a distance of about 0.5-2.5 nm as measured from the atom on each of Li and L2 to which the linker is attached. Further, without wishing to be bound by any particular theory, it is believed that such positioning of L1 and L2 relative to each other cannot be achieved with rigid linear linkers. For example, AZD5582 comprises a diyne linker having the structure

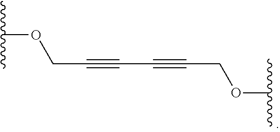 [SMILES: *OCC#CC#CCO*]

See Hennessy et al., J. Med. Chem. 2013, 56, 9897-9919. Hennessy et al. report that the linker should have minimal steric requirements to prevent disruption of critical binding interactions with the target protein. Hennessy et al. further report that a fully saturated linker (i.e.,

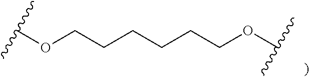 [SMILES: *OCCCCCCO*]

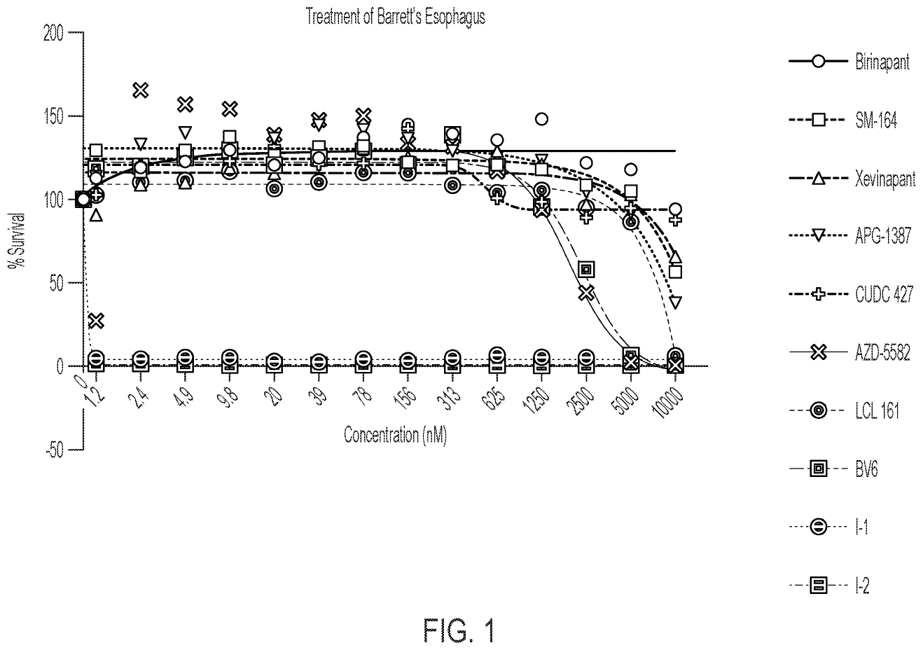

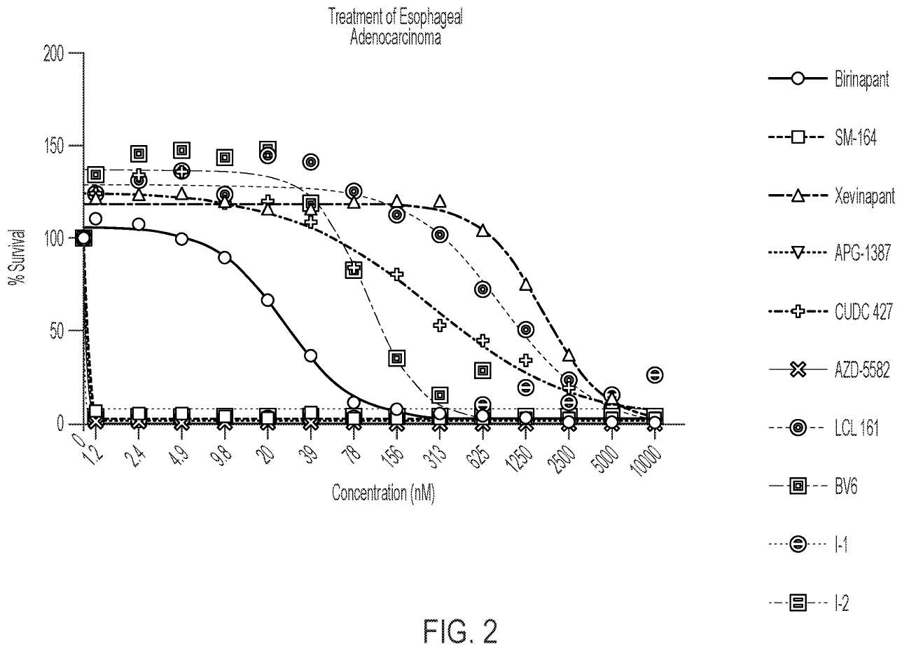

did not result in any appreciable change in the cellular potency relative to compounds such as AZD5582. Hennessy et al. further surmise that shorter, less hydrophobic linkers render compounds less cell-permeable and therefore less potent in cell-based assays. In some embodiments, the present disclosure provides the insight that, despite the teachings of Hennessy, compounds comprising less rigid, more hydrophilic linkers, such as those described herein (e.g., compounds having a linker comprising squaramide) demonstrate improved activity as compared to compounds with rigid, hydrophobic linkers such as AZD5582. See, for example, FIGS. 1 and 2. Additionally, FIGS. 1 and 2 demonstrate that compounds of formula I are more potent than compounds having flexible, hydrophobic linkers such as SM-164 and BV6.

In some embodiments, it will be appreciated that compounds comprising less hydrophobic linkers have lower log Ps relative to compounds comprising more hydrophobic linkers. For example, the log P for AZD5582 is calculated to be 6.14, whereas the log P of compound I-1 is calculated to be 5.5.

Thus, the present disclosure encompasses the insight that compounds of formula I are uniquely potent against cancer cell lines due to the flexibility and hydrophilicity of squaramide linkers as described herein.

As generally defined above, L1 is a first ligand; and L2 is a second ligand. In some embodiments, L1 and L2 are the same. In some embodiments, L1 and L2 are different.

In some embodiments, a ligand (e.g., L1 or L2) refers to a moiety that binds to a protein, for example, at a ligand binding domain. In some embodiments, a ligand (e.g., L1 or L2) is a moiety that binds to an IAP. In some embodiments, an IAP is selected from NAIP, cIAP1, cIAP2, XIAP, Survivin, Apollon, ML-IAP and ILP2.

In some embodiments, L1 is or comprises a group selected from

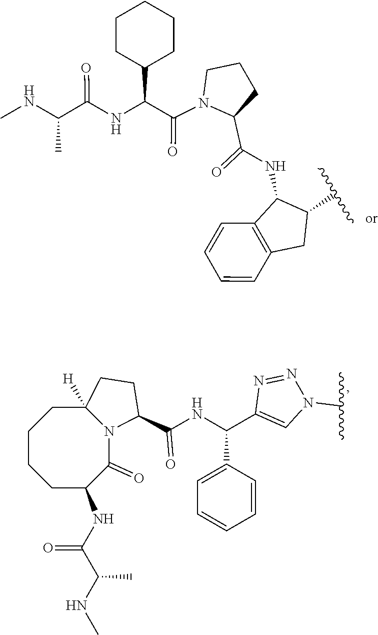 [SMILES: *[C@@H]1Cc2ccccc2[C@@H]1NC(=O)[C@@H]1CCCN1C(=O)[C@@H](NC(=O)[C@H](C)NC)C1CCCCC1 ; *n1cc([C@@H](NC(=O)[C@@H]2CC[C@@H]3CCCC[C@H](NC(=O)[C@H](C)NC)C(=O)N32)c2ccccc2)nn1]

In some embodiments, L2 is or comprises a group selected from

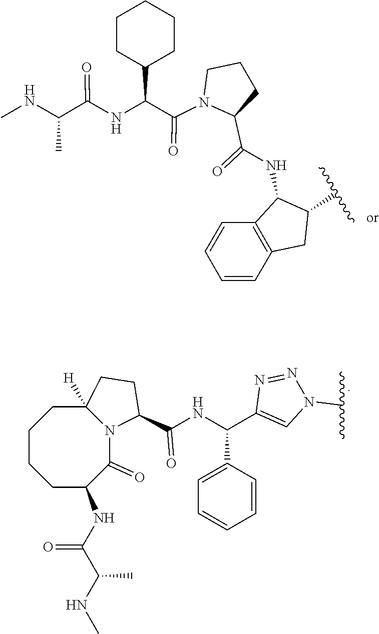 [SMILES: *[C@@H]1Cc2ccccc2[C@@H]1NC(=O)[C@@H]1CCCN1C(=O)[C@@H](NC(=O)[C@H](C)NC)C1CCCCC1 ; *n1cc([C@@H](NC(=O)[C@@H]2CC[C@@H]3CCCC[C@H](NC(=O)[C@H](C)NC)C(=O)N32)c2ccccc2)nn1]

As generally defined above, linker is a bivalent linker comprising

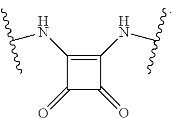 [SMILES: *Nc1c(N*)c(=O)c1=O]

In some embodiments, the linker is of formula X:

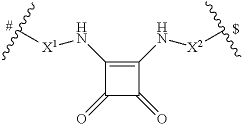

or a pharmaceutically acceptable salt thereof, wherein:
each of X¹ and X² is independently a covalent bond or an optionally substituted bivalent, saturated or partially unsaturated, straight or branched C₁₋₁₂ hydrocarbon chain, wherein 1-4 carbon atoms are optionally and independently replaced by —O—, —N(R)—, —C(O)—, —S—, —SO—, —SO₂—,
or -Cy-; each R is independently selected from hydrogen or an optionally substituted C₁₋₆ aliphatic; each -Cy- is independently an optionally substituted bivalent ring selected from a 3- to 8-membered carbocyclene, a 5- to 6-membered saturated or partially unsaturated heterocyclene having 1-3 heteroatoms independently selected from oxygen, nitrogen, or sulfur; phenylene; or a 5- to 6-membered heteroarylene having 1-3 heteroatoms independently selected from oxygen, nitrogen, or sulfur; #represents the point of attachment to L1; and $ represents the point of attachment to L2.

As generally defined above, each of X¹ and X² is independently a covalent bond or an optionally substituted bivalent, saturated or partially unsaturated, straight or branched C₁₋₁₂ hydrocarbon chain, wherein 1-4 carbon atoms are optionally and independently replaced by —O—, —N(R)—, —C(O)—, —S—, —SO—, —SO₂—, or -Cy-. In some embodiments, each of X¹ and X² is independently a covalent bond or an optionally substituted bivalent, saturated or partially unsaturated, straight or branched C₁₋₆ hydrocarbon chain, wherein 1-2 carbon atoms are optionally and independently replaced by —O—, —N(R)—, or —C(O)—. In some embodiments, each of X¹ and X² is independently a covalent bond or an optionally substituted bivalent, saturated or partially unsaturated, straight or branched C₁₋₈ hydrocarbon chain, wherein 1-2 carbon atoms are optionally and independently replaced by —O—, —N(R)—, or —C(O)—. In some embodiments, X¹ and X² are the same. In some embodiments, X¹ and X² are different.

In some embodiments, X¹ is a covalent bond. In some embodiments, X¹ is an optionally substituted bivalent, saturated or partially unsaturated, straight or branched C₁₋₆ hydrocarbon chain, wherein 1-2 carbon atoms are optionally and independently replaced by —O—, —N(R)—, or —C(O)—. In some embodiments, X¹ is an optionally substituted bivalent, saturated or partially unsaturated, straight or branched C₃₋₆ hydrocarbon chain, wherein 1-2 carbon atoms are optionally and independently replaced by —O— or —N(R)—. In some embodiments, X¹ is an optionally substituted bivalent, saturated or partially unsaturated, straight C₃₋₆ hydrocarbon chain, wherein 1-2 carbon atoms are optionally replaced by —O—.

In some embodiments, X¹ is an optionally substituted bivalent, saturated or partially unsaturated, straight C₃ hydrocarbon chain, wherein 1 carbon atom is optionally replaced by —O—. In some embodiments, X¹ is an optionally substituted bivalent, saturated, straight C₃ hydrocarbon chain, wherein 1 carbon atom is optionally replaced by —O—. In some embodiments, X¹ is an optionally substituted bivalent, saturated, straight C₃ hydrocarbon chain, wherein 1 carbon atom is replaced by —O—. In some embodiments, X¹ is an optionally substituted bivalent, saturated, straight C₃ hydrocarbon chain.

In some embodiments, X¹ is an optionally substituted bivalent, saturated or partially unsaturated, straight C₄ hydrocarbon chain, wherein 1 carbon atom is optionally replaced by —O—. In some embodiments, X¹ is an optionally substituted bivalent, saturated, straight C₄ hydrocarbon chain, wherein 1 carbon atom is optionally replaced by —O—. In some embodiments, X¹ is an optionally substituted bivalent, saturated, straight C₄ hydrocarbon chain.

In some embodiments, X¹ is an optionally substituted bivalent, saturated or partially unsaturated, straight C₅ hydrocarbon chain, wherein 1-2 carbon atoms are optionally replaced by —O—. In some embodiments, X¹ is an optionally substituted bivalent, saturated, straight C₅ hydrocarbon chain, wherein 1-2 carbon atoms are optionally replaced by —O—. In some embodiments, X¹ is an optionally substituted bivalent, saturated, straight C₅ hydrocarbon chain, wherein 1 carbon atom is optionally replaced by —O—. In some embodiments, X¹ is an optionally substituted bivalent, saturated, straight C₅ hydrocarbon chain, wherein 1 carbon atom is replaced by —O—. In some embodiments, X¹ is an optionally substituted bivalent, saturated, straight C₅ hydrocarbon chain.

In some embodiments, X¹ is an optionally substituted bivalent, saturated or partially unsaturated, straight C₆ hydrocarbon chain, wherein 1-2 carbon atoms are optionally replaced by —O—. In some embodiments, X¹ is an optionally substituted bivalent, saturated, straight C₆ hydrocarbon chain, wherein 1-2 carbon atoms are optionally replaced by —O—. In some embodiments, X¹ is an optionally substitute bivalent, saturated, straight C₆ hydrocarbon chain, wherein 1 carbon atom is replaced by —O—. In some embodiments, X¹ is an optionally substitute bivalent, saturated, straight C₆ hydrocarbon chain, wherein 2 carbon atoms are replaced by —O—.

In some embodiments, X¹ is an optionally substituted bivalent, saturated or partially unsaturated, straight C₇ hydrocarbon chain, wherein 1-2 carbon atoms are optionally replaced by —O—. In some embodiments, X¹ is an optionally substituted bivalent, saturated, straight C₇ hydrocarbon chain, wherein 1-2 carbon atoms are optionally replaced by —O—. In some embodiments, X¹ is an optionally substitute bivalent, saturated, straight C₇ hydrocarbon chain, wherein 1 carbon atom is replaced by —O—. In some embodiments, X¹ is an optionally substitute bivalent, saturated, straight C₇ hydrocarbon chain, wherein 2 carbon atoms are replaced by —O—.

In some embodiments, X¹ is an optionally substituted bivalent, saturated or partially unsaturated, straight C₈ hydrocarbon chain, wherein 1-2 carbon atoms are optionally replaced by —O—. In some embodiments, X¹ is an optionally substituted bivalent, saturated, straight C₈ hydrocarbon chain, wherein 1-2 carbon atoms are optionally replaced by —O—. In some embodiments, X is an optionally substitute bivalent, saturated, straight C₈ hydrocarbon chain, wherein 1 carbon atom is replaced by —O—. In some embodiments, X¹ is an optionally substitute bivalent, saturated, straight C₈ hydrocarbon chain, wherein 2 carbon atoms are replaced by —O—.

In some embodiments, X¹ is:
covalent bond,

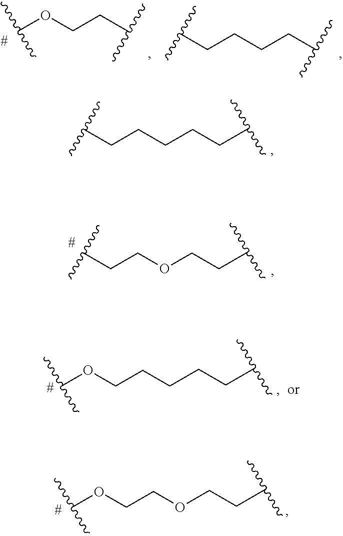 [SMILES: *CCO* ; *CCCC* ; *CCCCC* ; *CCOCC* ; *CCCCCO* ; *CCOCCO*]

wherein #represents the point of attachment to L1.

In some embodiments, X¹ is:
covalent bond,

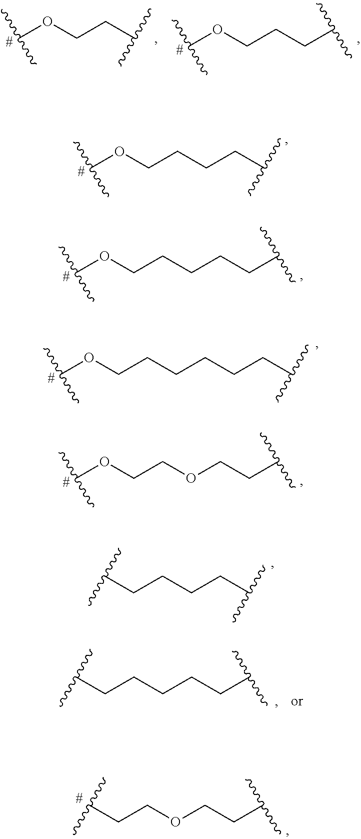

wherein #represents the point of attachment to Li.

In some embodiments, X¹ is

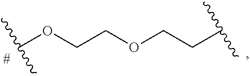 [SMILES: *CCOCCO*]

wherein #represents the point of attachment to L1.

In some embodiments, X¹ is

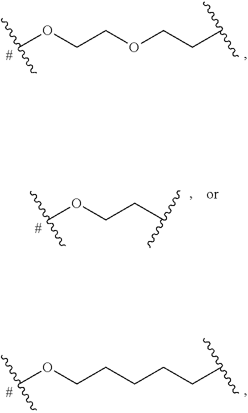 [SMILES: *CCOCCO* ; *CCO* ; *CCCCCO*]

wherein #represents the point of attachment to L1.

In some embodiments, X² is a covalent bond. In some embodiments, X² is an optionally substituted bivalent, saturated or partially unsaturated, straight or branched C₁₋₆ hydrocarbon chain, wherein 1-2 carbon atoms are optionally and independently replaced by —O—, —N(R)—, or —C(O)—. In some embodiments, X² is an optionally substituted bivalent, saturated or partially unsaturated, straight or branched C₃₋₆ hydrocarbon chain, wherein 1-2 carbon atoms are optionally and independently replaced by —O— or —N(R)—. In some embodiments, X² is an optionally substituted bivalent, saturated or partially unsaturated, straight C₃₋₆ hydrocarbon chain, wherein 1-2 carbon atoms are optionally replaced by —O—.

In some embodiments, X² is an optionally substituted bivalent, saturated or partially unsaturated, straight C₃ hydrocarbon chain, wherein 1 carbon atom is optionally replaced by —O—. In some embodiments, X² is an optionally substituted bivalent, saturated, straight C₃ hydrocarbon chain, wherein 1 carbon atom is optionally replaced by —O—. In some embodiments, X² is an optionally substituted bivalent, saturated, straight C₃ hydrocarbon chain, wherein 1 carbon atom is replaced by —O—. In some embodiments, X² is an optionally substituted bivalent, saturated, straight C₃ hydrocarbon chain.

In some embodiments, X² is an optionally substituted bivalent, saturated or partially unsaturated, straight C₄ hydrocarbon chain, wherein 1 carbon atom is optionally replaced by —O—. In some embodiments, X² is an optionally substituted bivalent, saturated, straight C₄ hydrocarbon chain, wherein 1 carbon atom is optionally replaced by —O—. In some embodiments, X² is an optionally substituted bivalent, saturated, straight C₄ hydrocarbon chain.

In some embodiments, X² is an optionally substituted bivalent, saturated or partially unsaturated, straight C₅ hydrocarbon chain, wherein 1-2 carbon atoms are optionally replaced by —O—. In some embodiments, X² is an optionally substituted bivalent, saturated, straight C₅ hydrocarbon chain, wherein 1-2 carbon atoms are optionally replaced by —O—. In some embodiments, X² is an optionally substituted bivalent, saturated, straight C₅ hydrocarbon chain, wherein 1 carbon atom is optionally replaced by —O—. In some embodiments, X² is an optionally substituted bivalent, saturated, straight C₅ hydrocarbon chain, wherein 1 carbon atom is replaced by —O—. In some embodiments, X² is an optionally substituted bivalent, saturated, straight C₅ hydrocarbon chain.

In some embodiments, X² is an optionally substituted bivalent, saturated or partially unsaturated, straight C₆ hydrocarbon chain, wherein 1-2 carbon atoms are optionally replaced by —O—. In some embodiments, X² is an optionally substituted bivalent, saturated, straight C₆ hydrocarbon chain, wherein 1-2 carbon atoms are optionally replaced by —O—. In some embodiments, X² is an optionally substitute bivalent, saturated, straight C₆ hydrocarbon chain, wherein 1 carbon atom is replaced by —O—. In some embodiments, X² is an optionally substitute bivalent, saturated, straight C₆ hydrocarbon chain, wherein 2 carbon atoms are replaced by —O—.

In some embodiments, X² is an optionally substituted bivalent, saturated or partially unsaturated, straight C₇ hydrocarbon chain, wherein 1-2 carbon atoms are optionally replaced by —O—. In some embodiments, X² is an optionally substituted bivalent, saturated, straight C₇ hydrocarbon chain, wherein 1-2 carbon atoms are optionally replaced by —O—. In some embodiments, X² is an optionally substitute bivalent, saturated, straight C₇ hydrocarbon chain, wherein 1 carbon atom is replaced by —O—. In some embodiments, X² is an optionally substitute bivalent, saturated, straight C₇ hydrocarbon chain, wherein 2 carbon atoms are replaced by —O—.

In some embodiments, X² is an optionally substituted bivalent, saturated or partially unsaturated, straight C₈ hydrocarbon chain, wherein 1-2 carbon atoms are optionally replaced by —O—. In some embodiments, X² is an optionally substituted bivalent, saturated, straight C₈ hydrocarbon chain, wherein 1-2 carbon atoms are optionally replaced by —O—. In some embodiments, X² is an optionally substitute bivalent, saturated, straight Cₛ hydrocarbon chain, wherein 1 carbon atom is replaced by —O—. In some embodiments, X² is an optionally substitute bivalent, saturated, straight C₈ hydrocarbon chain, wherein 2 carbon atoms are replaced by —O—.

In some embodiments, X² is:
covalent bond,

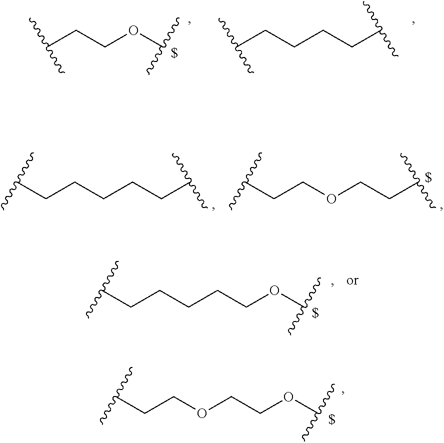 [SMILES: *CCO* ; *CCCC* ; *CCCCC* ; *CCOCC* ; *CCCCCO* ; *CCOCCO*]

wherein $ represents the point of attachment to L2.

In some embodiments, X² is:
covalent bond,

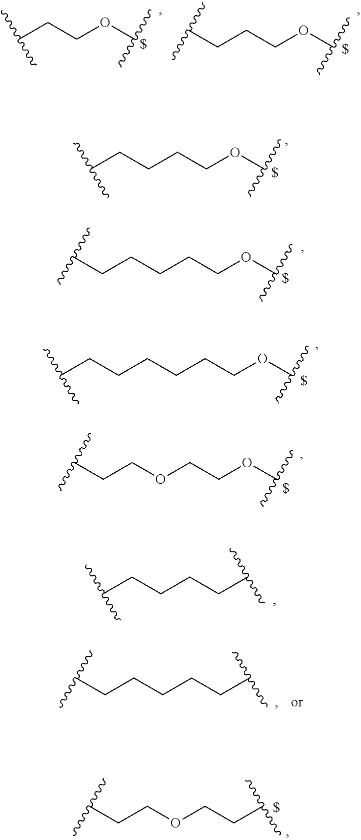

wherein $ represents the point of attachment to L2.

In some embodiments, X² is

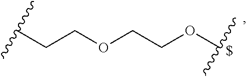 [SMILES: *CCOCCO*]

wherein #represents the point of attachment to L2.

In some embodiments, X² is

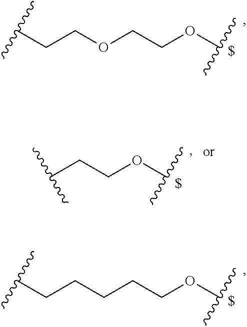 [SMILES: *CCOCCO* ; *CCO* ; *CCCCCO*]

wherein #represents the point of attachment to L2.

As generally defined above, each R is independently selected from hydrogen or an optionally substituted C₁₋₆ aliphatic. In some embodiments R is hydrogen. In some embodiments, R is optionally substituted C₁₋₆ aliphatic.

As generally defined above, each -Cy- is independently an optionally substituted bivalent ring selected from a 3- to 8-membered carbocyclene, a 5- to 6-membered saturated or partially unsaturated heterocyclene having 1-3 heteroatoms independently selected from oxygen, nitrogen, or sulfur; phenylene; or a 5- to 6-membered heteroarylene having 1-3 heteroatoms independently selected from oxygen, nitrogen, or sulfur. In some embodiments, -Cy- is a 3- to 8-membered carbocyclene. In some embodiments, -Cy- is a 5- to 6-membered saturated or partially unsaturated heterocyclene having 1-3 heteroatoms independently selected from oxygen, nitrogen, or sulfur. In some embodiments, -Cy- is phenylene. In some embodiments, -Cy- is a 5- to 6-membered heteroarylene having 1-3 heteroatoms independently selected from oxygen, nitrogen, or sulfur.

In some embodiments, the present disclosure provides a compound of formula I-a, I-b, or I-c.

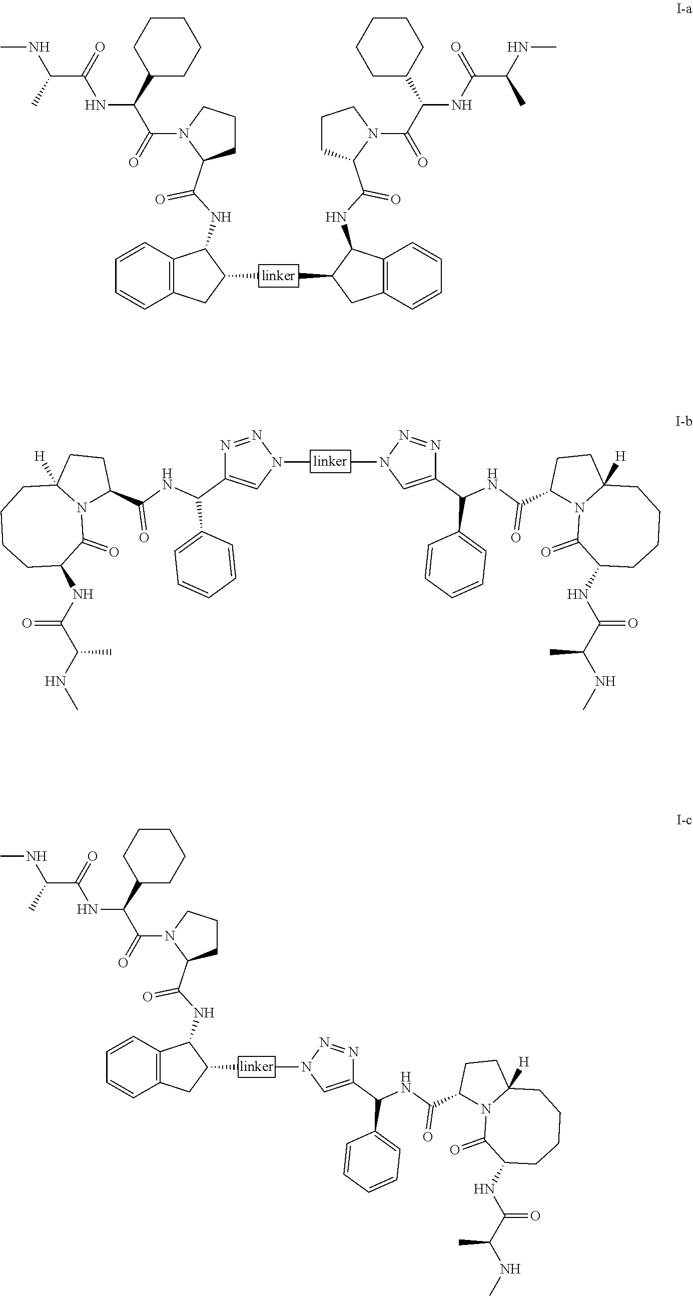

or a pharmaceutically acceptable salt thereof, wherein linker is as defined above and described herein.

In some embodiments, it will be appreciated that the linker serves to position L1 and L2 at a particular distance relative to each other (e.g., between about 0.5-2.5 nm).

In certain embodiments of formula I-a, the linker is sufficient to position L1 and L2 at a distance of about 0.5-2.5 nm between the C1 carbon atoms of the respective indanyl groups (indicated by * below):

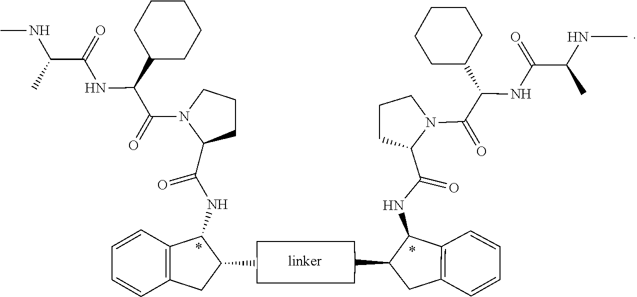

In some embodiments of formula I-a, the linker is sufficient to position L1 and L2 at a distance of about 0.7-2.2 nm between the C1 carbon atoms of the respective indanyl groups. In some embodiments of formula I-a, the linker is sufficient to position L1 and L2 at a distance of about 1.0-2.2 nm between the C1 carbon atoms of the respective indanyl groups. In some embodiments of formula I-a, the linker is sufficient to position L1 and L2 at a distance of about 1.4-2.2 nm between the C1 carbon atoms of the respective indanyl groups. In some embodiments of formula I-a, the linker is sufficient to position L1 and L2 at a distance of about 1.8-2.2 nm between the C1 carbon atoms of the respective indanyl groups. In some embodiments of formula I-a, the linker is sufficient to position L1 and L2 at a distance of about 2.0-2.2 nm between the C1 carbon atoms of the respective indanyl groups. In some embodiments of formula I-a, the linker is sufficient to position L1 and L2 at a distance of about 1.2-1.8 nm between the C1 carbon atoms of the respective indanyl groups. In some embodiments of formula I-a, the linker is sufficient to position L1 and L2 at a distance of about 1.3-1.5 nm between the C1 carbon atoms of the respective indanyl groups. In some embodiments of formula I-a, the linker is sufficient to position L1 and L2 at a distance of about 0.7-1.5 nm between the C1 carbon atoms of the respective indanyl groups. In some embodiments of formula I-a, the linker is sufficient to position L1 and L2 at a distance of about 0.7-1.0 nm between the C1 carbon atoms of the respective indanyl groups. In some embodiments of formula I-a, the linker is sufficient to position LI and L2 at a distance of about 0.7-0.8 nm between the C1 carbon atoms of the respective indanyl groups. In some embodiments of formula I-a, the linker is sufficient to position L1 and L2 at a distance of about 0.7-0.8, 1.4-1.5, or 2.0-2.2 nm between the C1 carbon atoms of the respective indanyl groups. In some embodiments of formula I-a, the linker is sufficient to position L1 and L2 at a distance of about 0.7, 1.5, or 2.1 nm between the C1 carbon atoms of the respective indanyl groups.

In certain embodiments of formula 1-b, the linker is sufficient to position LI and L2 at a distance of about 1.5-2.5 nm between the respective benzylic carbon atoms (indicated by * below):

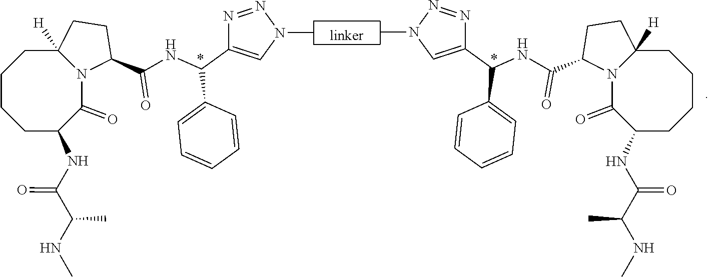

In some embodiments of formula I-b, the linker is sufficient to position L1 and L2 at a distance of about 1.9-2.2 nm between the respective benzylic carbon atoms. In some embodiments of formula I-b, the linker is sufficient to position L1 and L2 at a distance of about 1.9-2.0 nm between the respective benzylic carbon atoms. In some embodiments of formula I-b, the linker is sufficient to position L1 and L2 at a distance of about 2.0-2.2 nm between the respective benzylic carbon atoms. In some embodiments of formula I-b, the linker is sufficient to position L1 and L2 at a distance of about 2.1-2.2 nm between the respective benzylic carbon atoms. In some embodiments of formula I-b, the linker is sufficient to position L1 and L2 at a distance of about 1.9 or 2.1 nm between the respective benzylic carbon atoms.

In certain embodiments of formula I-c, the linker is sufficient to position L1 and L2 at a distance of about 1.5-2.5 nm between the indanyl carbon atom of L1 and the benzylic carbon atom of L2 (indicated by * below):

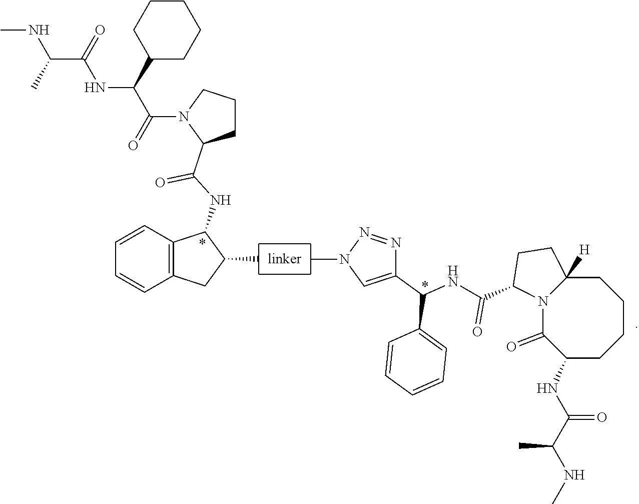

In some embodiments of formula I-c, the linker is sufficient to position L1 and L2 at a distance of about 1.7-2.3 nm between the indanyl carbon atom of L1 and the benzylic carbon atom of L2. In some embodiments of formula I-c, the linker is sufficient to position L1 and L2 at a distance of about 1.9-2.1 nm between the indanyl carbon atom of L1 and the benzylic carbon atom of L2. In some embodiments of formula I-c, the linker is sufficient to position L1 and L2 at a distance of about 1.9-2.1 nm between the indanyl carbon atom of L1 and the benzylic carbon atom of L2.

In some embodiments, a compound of Formula I is selected from:

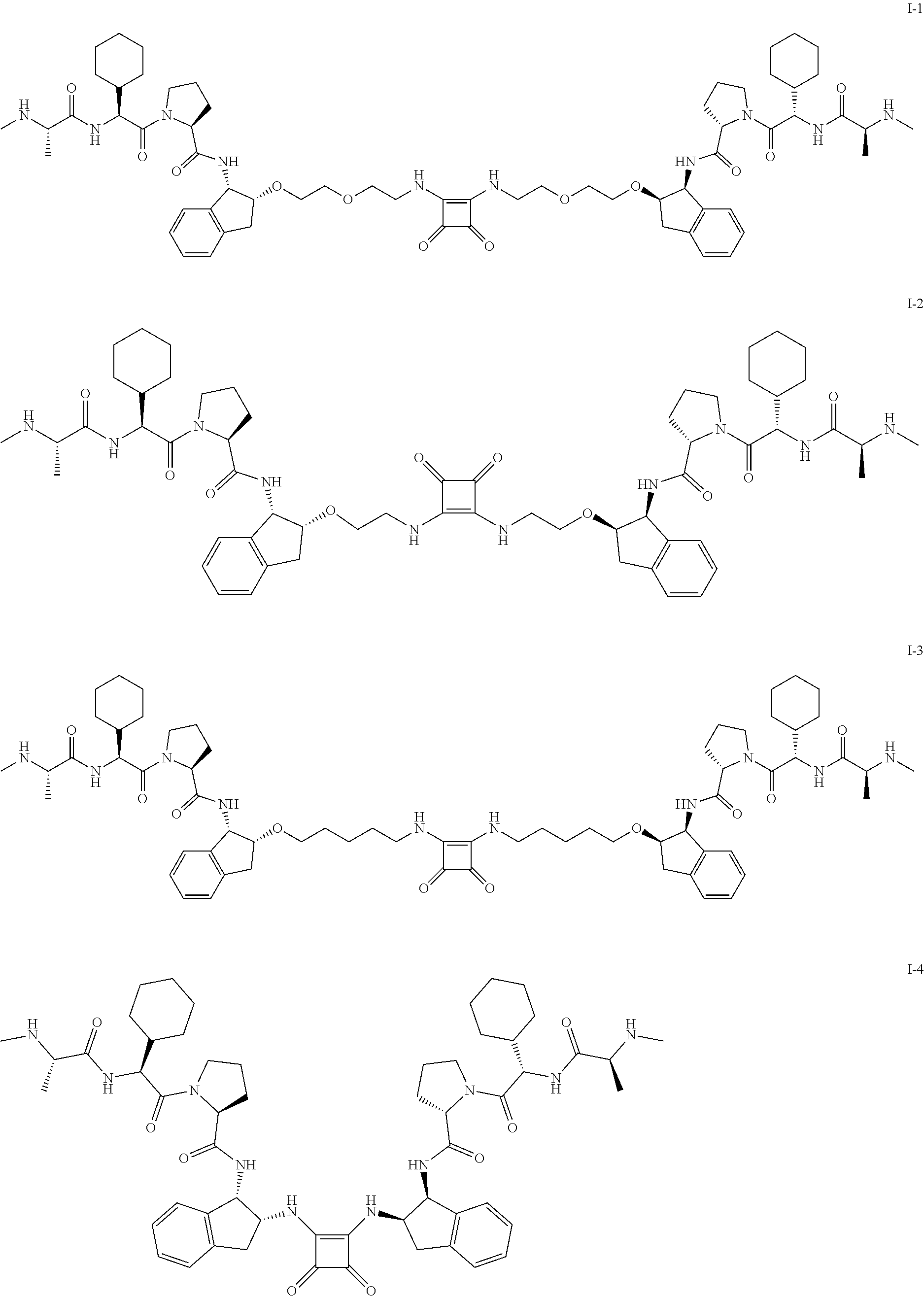

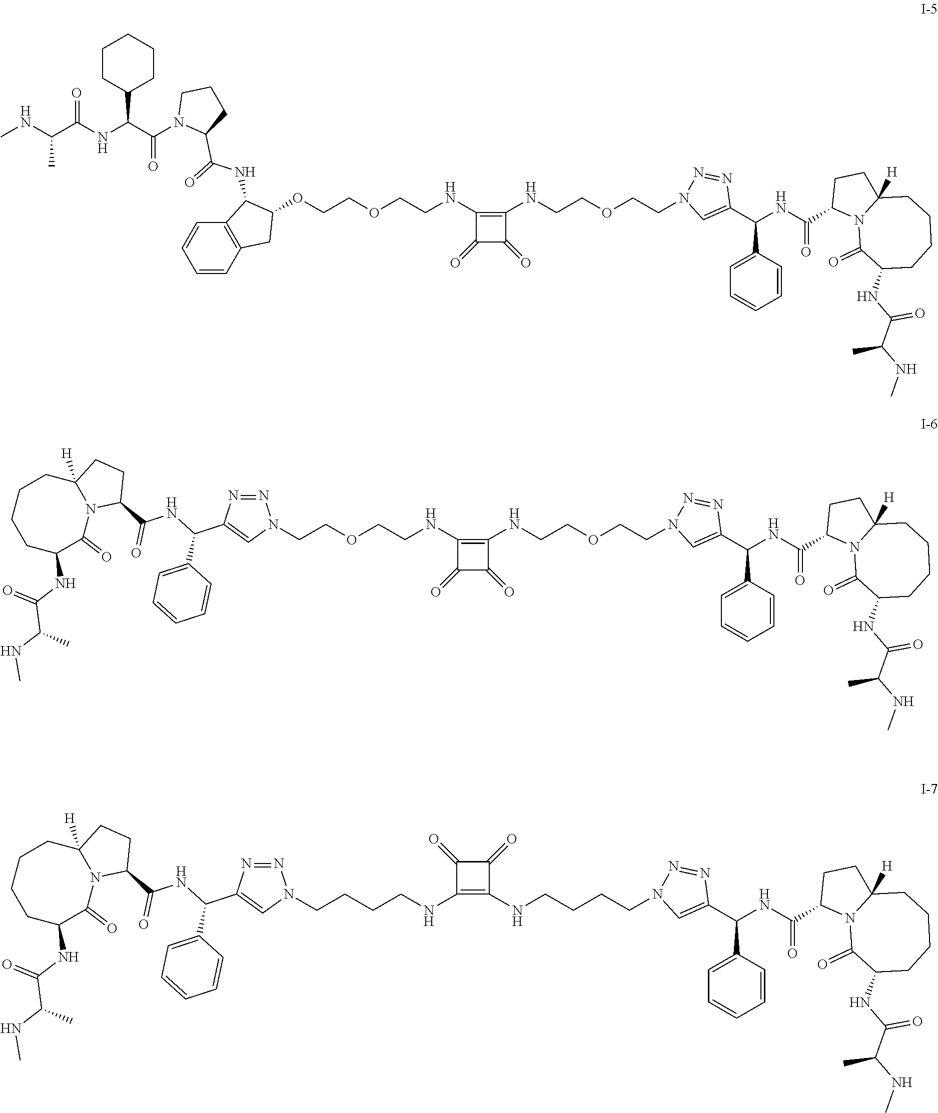

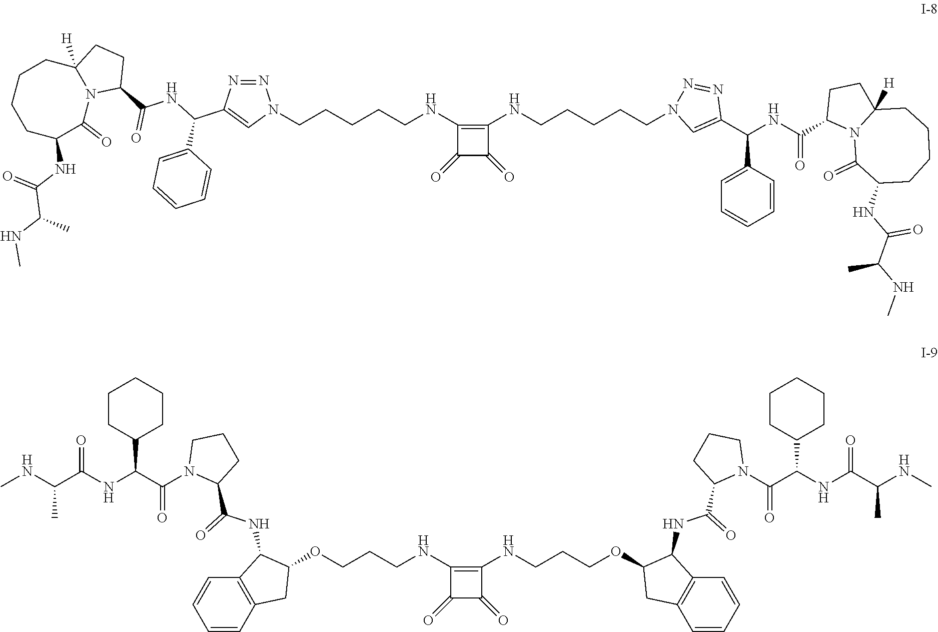

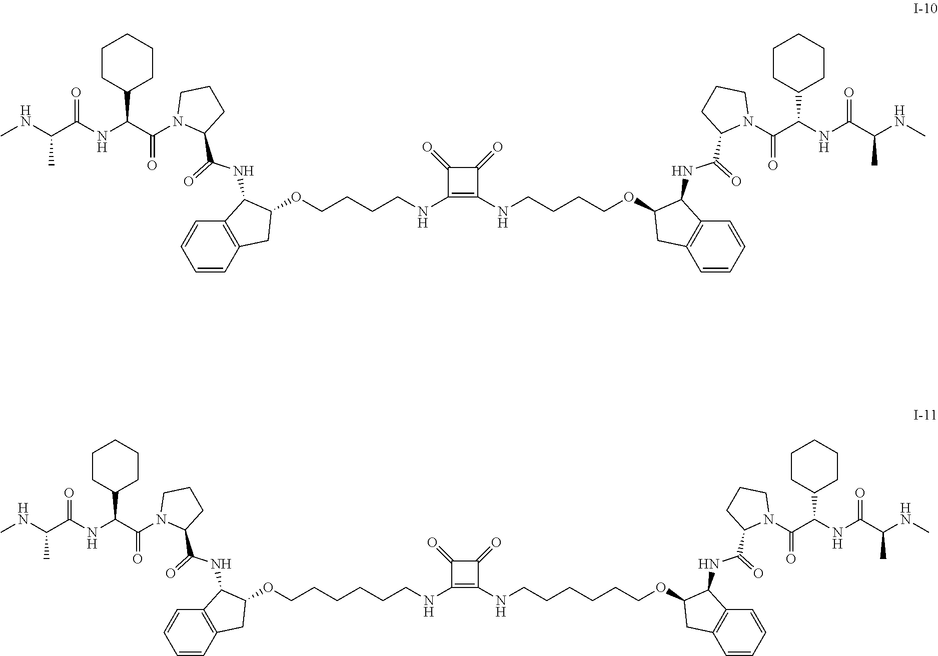

or a pharmaceutically acceptable salt thereof.

| Compound | logP | PSA |
|---|---|---|
| I-1 | 5.49 | 276.20 |
| I-2 | 5.46 | 257.74 |
| I-3 | 7.80 | 257.74 |
| I-4 | 5.42 | 239.28 |
| I-5 | 4.55 | 297.68 |
| I-6 | 3.61 | 319.16 |
| I-7 | 5.14 | 300.70 |
| I-8 | 5.92 | 300.70 |
| I-9 | 6.24 | 257.74 |
| I-10 | 7.02 | 257.74 |
| I-11 | 8.58 | 257.74 |

### 4. Uses, Formulation, and Administration

### Pharmaceutically Acceptable Compositions

According to another embodiment, the present disclosure provides a composition comprising a compound described herein or a pharmaceutically acceptable salt thereof and a pharmaceutically acceptable carrier, adjuvant, or vehicle. In certain embodiments, the amount of compound in compositions described herein is such that it is effective to measurably inhibit activity of an IAP (e.g., BIRC1/NAIP, BIRC2/cIAP1, BIRC3/cIAP2, BIRC4/XIAP, BIRC5/Survivin, BIRC6/Apollon, BIRC7/ML-IAP and BIRC8/ILP2), or a mutant thereof, in a biological sample or in a patient. In certain embodiments, a composition described herein is formulated for administration to a patient in need of such composition. In some embodiments, a composition described herein is formulated for oral administration to a patient.

Compounds and compositions, according to method of the present disclosure, are administered using any amount and any route of administration effective for treating or lessening the severity of a disorder provided herein (i.e., an IAP-mediated disease or disorder). The exact amount required will vary from subject to subject, depending on the species, age, and general condition of the subject, the severity of the infection, the particular agent, its mode of administration, and the like. Compounds described herein are preferably formulated in unit dosage form for ease of administration and uniformity of dosage.

Compositions of the present disclosure may be administered orally, parenterally, by inhalation spray, topically, rectally, nasally, buccally, vaginally, intraperitoneally, intracisternally or via an implanted reservoir. In some embodiments, the compositions are administered orally, intraperitoneally or intravenously.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present disclosure, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

In some embodiments, provided pharmaceutically acceptable compositions are formulated for oral administration. Such formulations may be administered with or without food. In some embodiments, pharmaceutically acceptable compositions described herein are administered without food. In other embodiments, pharmaceutically acceptable compositions described herein are administered with food. Pharmaceutically acceptable compositions described herein may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and/or i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Alternatively, pharmaceutically acceptable compositions described herein may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Compositions for rectal or vaginal administration are preferably suppositories which can be prepared by mixing the compounds described herein with suitable non-irritating excipients or carriers such as cocoa butter, polyethylene glycol or a suppository wax which are solid at ambient temperature but liquid at body temperature and therefore melt in the rectum or vaginal cavity and release the active compound.

Pharmaceutically acceptable compositions described herein may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds described herein include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions described herein may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. In some embodiments pharmaceutically acceptable compositions described herein can be administered by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurised container, pump, spray, atomizer, or nebuliser contains a solution or suspension of the compound(s) of the disclosure comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Dosage forms for topical or transdermal administration of a compound disclosed herein include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this disclosure. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

### Uses of Compounds and Pharmaceutically Acceptable Compositions

IAP (Inhibitor of apoptosis) proteins, a family of anti-apoptotic proteins, have an important role in evasion of apoptosis, as they can both block apoptosis-signaling pathways and promote survival. Eight members of this family have been described in humans (BIRC1/NAIP, BIRC2/cIAP1, BIRC3/cIAP2, BIRC4/XIAP, BIRC5/Survivin, BIRC6/Apollon, BIRC7/ML-IAP and BIRC8/ILP2). In certain embodiments, the agent is an IAP Inhibitor (i.e., an IAP Antagonist). Exemplary IAP Inhibitors include XIAP inhibitors, CIAP inhibitors, and agents acting as dual XIAP and CIAP inhibitors.

Exemplary IAP inhibitors and antagonists include Birinapant (a bivalent Smac mimetic, which is a potent antagonist for XIAP and cIAP1 with Kds of 45 nM and less than 1 nM, respectively), LCL 161 Inhibitor (an IAP inhibitor which inhibits XIAP and cIAP1 with IC₅₀'s of 35 and 0.4 nM), AZD5582 (AZD5582 an IAP antagonist which binds to the BIR3 domains cIAP1, cIAP2, and XIAP), SM-164 (a cell-permeable Smac mimetic compound that binds to XIAP protein containing both the BIR2 and BIR3 domains with an IC₅₀ value of 1.39 nM and functions as an extremely potent antagonist of XIAP), BV6 (an antagonist of cIAP1 and XIAP), Xevinapant (or AT-406, is a potent and orally bioavailable Smac mimetic and an antagonist of IAPs, and it binds to XIAP, cIAP1, and cIAP2 proteins), GDC-0152 (a potent IAPs inhibitor, and binds to the BIR3 domains of XIAP, cIAP1, cIAP2 and the BIR domain of ML-IAP), ASTX660 (an orally bioavailable dual antagonist of cIAPs and XIAPs), CUDC-427 (a potent second-generation pan-selective IAP antagonist), Embelin (or Embelic acid, a potent, nonpeptidic XIAP inhibitor). APG-1387 (a bivalent SMAC mimetic and an IAP antagonist, blocks the activity of IAPs family proteins (XIAP, cIAP-1, cIAP-2, and ML-IAP), MX69 (an inhibitor of MDM2/XIAP), MVI, Polygalacin D, UC-112, AZD5582 dihydrochloride, HY-125378m Tolinapant (ASTX660) and SBP-0636457.

In certain embodiments, the IAP inhibitor is a selective XIAP inhibitor (having an IC₅₀ for XIAP inhibition at least 10-fold less than the IC₅₀ for CIAP inhibition, and more preferably at least 20, 50 or 100-fold less), such as SM-164.

In some embodiments, compounds disclosed herein bind to one or more IAPs (e.g., NAIP, cIAP1, cIAP2, XIAP, Survivin, Apollon, ML-IAP, or ILP2). In some embodiments, compounds disclosed herein inhibitor activity or one or more IAPs (e.g., NAIP, cIAP1, cIAP2, XIAP, Survivin, Apollon, ML-IAP, or ILP2).

The activity of a compound described herein as an inhibitor of one or more IAPs (e.g., NAIP, cIAP1, cIAP2, XIAP, Survivin, Apollon, ML-IAP, or ILP2), or variants or mutants thereof, can be assayed in vitro, in vivo, or in a cell line. In vitro assays include assays that determine inhibition of one or more IAPs (e.g., NAIP, cIAP1, cIAP2, XIAP, Survivin, Apollon, MLL-IAP, or ILP2), or variants or mutants thereof. Alternate in vitro assays quantitate the ability of the inhibitor to bind to one or more IAPs (e.g., NAIP, cIAP1, cIAP2, XIAP, Survivin, Apollon, ML-IAP, or ILP2), or variants or mutants thereof. Detailed conditions for assaying a compound described herein as an inhibitor of one or more IAPs (e.g., NAIP, cIAP1, cIAP2, XIAP, Survivin, Apollon, ML-IAP, or ILP2), or variants or mutants thereof, are well known in the art and set forth in the Examples below.

The provided compounds are inhibitors of one or more IAPs (e.g., NAIP, cIAP1, cIAP2, XIAP, Survivin, Apollon, ML-IAP, or ILP2), or variants or mutants thereof, and are therefore useful for treating one or more disorders associated with activity of one or more IAPs (e.g., NAIP, cIAPL, cIAP2, XIAP, Survivin, Apollon, ML-IAP, or ILP2). Thus, in some aspects and embodiments, the present disclosure provides a method for treating an IAP-mediated disease, disorder, or condition comprising the step of administering to a patient in need thereof a compound of the present disclosure, or pharmaceutically acceptable composition thereof.

In some embodiments, the present disclosure provides a method of inhibiting one or more IAPs (e.g., NAIP, cIAP1, cIAP2, XIAP, Survivin, Apollon, ML-IAP, or ILP2), or variants or mutants thereof, comprising contacting a cell with a provided compound.

As used herein, a “disease or disorder associated with IAPs” or, alternatively, “a IAP-mediated disease or disorder” means any disease or other deleterious condition in which an IAP, or a mutant thereof, is known or suspected to play a role. In some embodiments, an IAP is selected from BIRC1/NAIP, BIRC2/cIAP1, BIRC3/cIAP2, BIRC4/XIAP, BIRC5/Survivin, BIRC6/Apollon, BIRC7/ML-IAP and BIRC8/ILP2. Accordingly, another embodiment of the present disclosure relates to treating or lessening the severity of one or more diseases in which one or more IAPs (e.g., NAIP, cIAP1, cIAP2, XIAP, Survivin, Apollon, ML-IAP, or ILP2), or variants or mutants thereof, is known or suspected to play a role.

In some embodiments, the present disclosure provides methods of treating, reducing the severity of, delaying the onset of, or inhibiting the progress of a disease or disorder, or one or more symptoms thereof of a disease or disorder associated with one or more IAPs (e.g., NAIP, cIAP1, cIAP2, XIAP, Survivin, Apollon, ML-IAP, or ILP2), or variants or mutants thereof, comprising the step of administering to a patient in need thereof a therapeutically effective a compound of the present disclosure, or pharmaceutically acceptable composition thereof. In some embodiments, the present disclosure provides methods of treating, reducing the severity of, delaying the onset of, or inhibiting the progress of a disease or disorder, or one or more symptoms thereof of a disease or disorder in which inhibition or antagonizing of activity of one or more IAPs (e.g., NAIP, cIAP1, cIAP2, XIAP, Survivin, Apollon, ML-IAP, or ILP2), or variants or mutants thereof, is beneficial comprising the step of administering to a patient in need thereof a compound described herein, or pharmaceutically acceptable composition thereof.

In some aspects and embodiments, the provided compounds therefore are useful for the treatment of disorders responsive to induction of apoptotic cell death, e.g., disorders characterized by dysregulation of apoptosis, including hyperproliferative diseases.

In some embodiments, the present disclosure provides a method for treating or lessening the severity of a cancer comprising administering to a patient in need thereof, a compound as described herein, or a pharmaceutical salt or composition thereof. In some embodiments, the cancer is breast cancer, prostate cancer, lymphoma, skin cancer, pancreatic cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma.

In some embodiments, the present disclosure provides a method for treating or lessening the severity of a pulmonary disease, disorder, or condition comprising administering to a patient in need thereof, a compound as described herein, or a pharmaceutical salt or composition thereof. In some embodiments, a pulmonary disease comprises an inflammatory disease or condition. In some embodiments, a pulmonary disease, disorder, or condition is chronic obstructive pulmonary disease (COPD), cystic fibrosis, airway inflammation, allergy(ies), asthma, impeded respiration, Acute respiratory distress syndrome, pulmonary hypertension, lung inflammation, bronchitis, airway obstruction, bronchoconstriction, microbial infection, viral infection (such as SARS), idiopathic pulmonary fibrosis, Asthma, bronchopulmonary dysplasia (BPD), chronic bronchitis or emphysema, or COVID-19.

Exemplary Methods of Manufacturing Compounds of this Present Disclosure

In some aspects, compounds of the present disclosure may be made by a variety of ways well-known to those skilled in the art of organic synthesis. By way of example, compounds of the present invention can be synthesized using methods described below and/or as described in WO 2007/130626A2 and WO 2010/142994A1, together with synthetic methods known in the art of synthetic organic chemistry, or variations thereon as appreciated by those skilled in the art. Compounds of the present application can be synthesized by the following steps outlined in the General Schemes below. Starting materials are either commercially available or made by known procedures in the reported literature.

In some embodiments, a compound of Formula I-a can be obtained using the method of General Scheme A below:

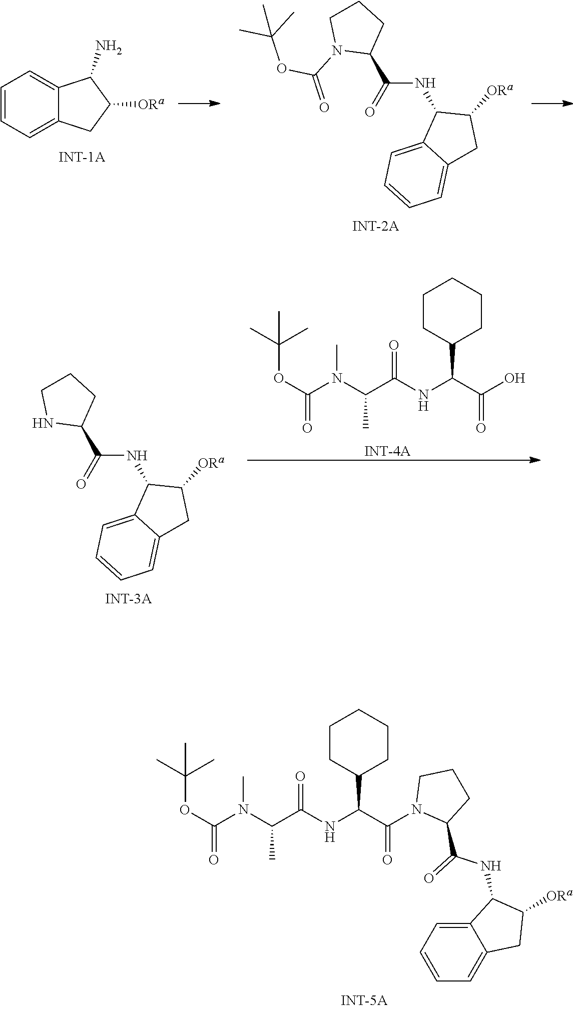

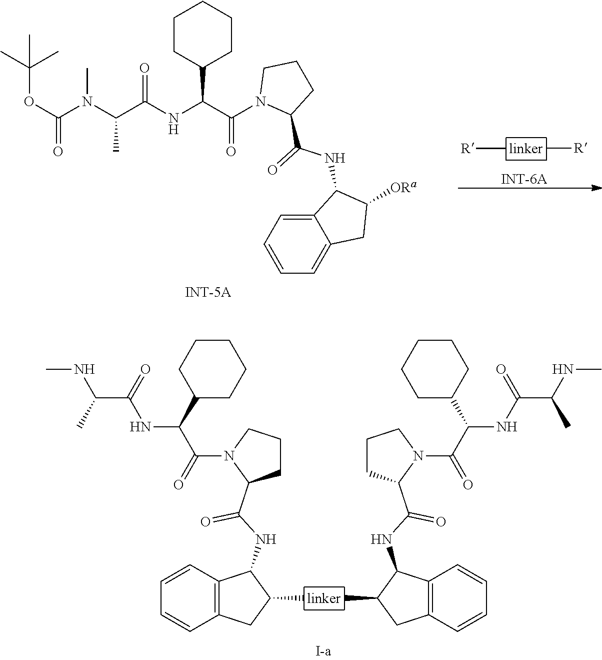

In some embodiments, it will be appreciated that compounds of formula I-a may be obtained as shown in General Scheme A using reagents and reaction conditions well known in the art, e.g., as described in WO 2010/142994A1. In some embodiments, R^{a} is a suitable moiety (or protected analog) where treatment with INT-6A affords a compound of formula I-a. In some embodiments, R′ is a suitable moiety (or protected analog) where treatment with INT-5A affords a compound of formula I-a.

In some embodiments, a compound of Formula 1-b can be obtained using the method of General Scheme B below:

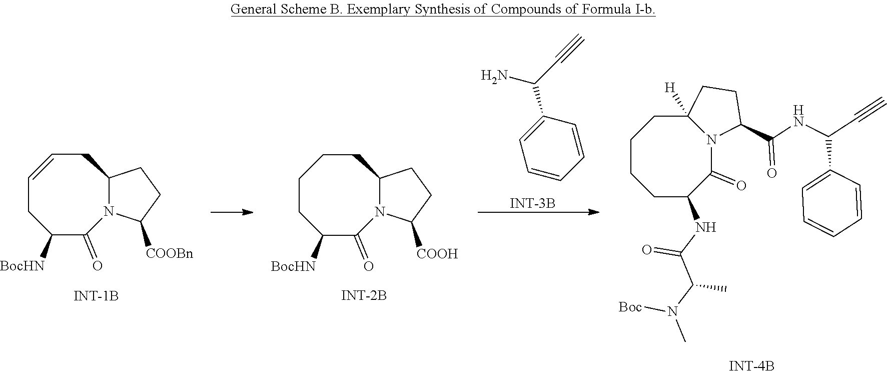

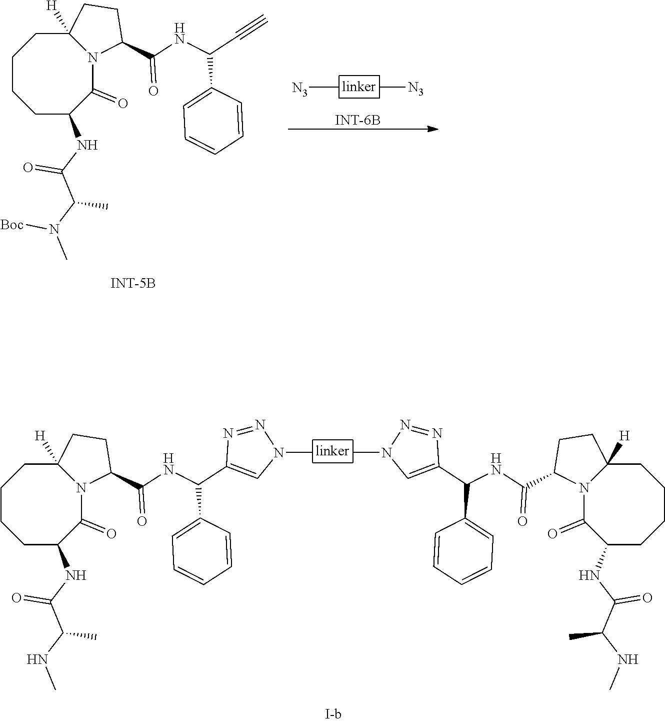

In some embodiments, it will be appreciated that compounds of formula I-b may be obtained as shown in General Scheme B using reagents and reaction conditions well known in the art, e.g., as described in WO 2007/130626A2.

In some embodiments, a compound of Formula I-c can be obtained using the method of General Scheme C below:

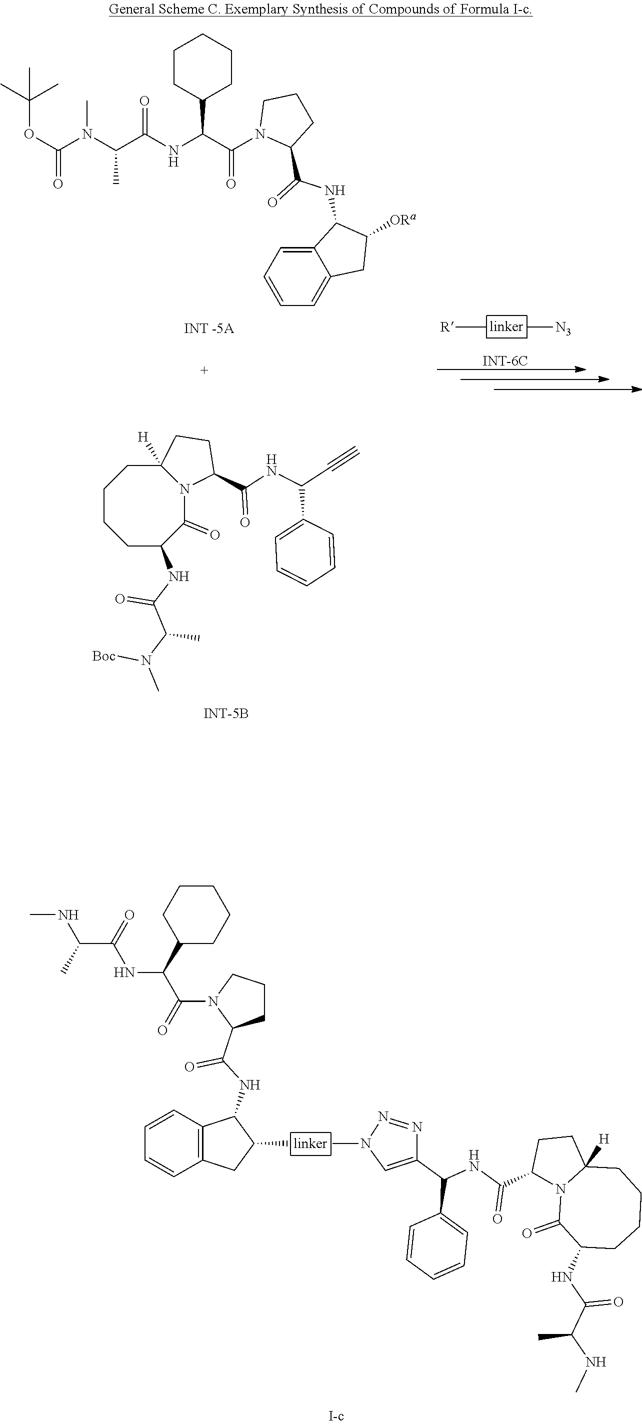

In some embodiments, it will be appreciated that compounds of formula I-c may be obtained as shown in General Scheme C using reagents and reaction conditions well known in the art, e.g., as described in WO 2007/130626A2 and WO 2010/142994A1. In some embodiments, R^{a} is a suitable moiety (or protected analog) where treatment with INT-6C affords a compound of formula I-c. In some embodiments, R′ is a suitable moiety (or protected analog) where treatment with INT-5A affords a compound of formula I-c.

### EXEMPLIFICATION

As depicted in the Examples below, in certain exemplary embodiments, compounds are prepared according to the following general procedures. It will be appreciated that, although the general methods depict the synthesis of certain compounds of the present disclosure, the following general methods, and other methods known to one of ordinary skill in the art, can be applied to all compounds and subclasses and species of each of these compounds, as described herein.

### Example 1: Treatment of Pulmonary Disease

The present example demonstrates treatment of a pulmonary disease with compounds described herein. Specifically, the present example demonstrates treatment of Barrett's esophagus (BE; precursor lesion for esophageal adenocarcinoma (EAC)). Cells isolated from individuals diagnosed with or suffering from Barrett's esophagus were treated with the indicated compounds. The Barrett's esophagus cells are particularly sensitive to compounds I-1 and I-2 as described herein (see FIG. 1). The Barrett's esophagus cells were significantly more sensitive to compounds I-1 and I-2 compared to the other compounds as listed in Table 1.

TABLE 1

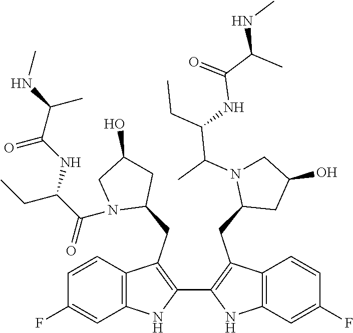 [SMILES: CC[C@H](NC(=O)[C@H](C)NC)C(=O)N1C[C@@H](O)C[C@H]1Cc1c(-c2[nH]c3cc(F)ccc3c2C[C@@H]2C[C@H](O)CN2C(C)[C@H](CC)NC(=O)[C@H](C)NC)[nH]c2cc(F)ccc12]

Birinapant

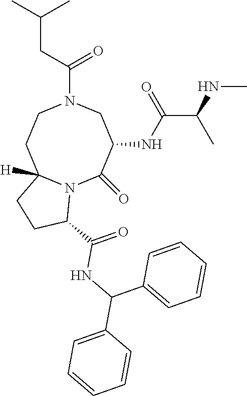 [SMILES: CN[C@@H](C)C(=O)N[C@H]1CN(C(=O)CC(C)C)CC[C@H]2CC[C@@H](C(=O)NC(c3ccccc3)c3ccccc3)N2C1=O]

Xevinapant

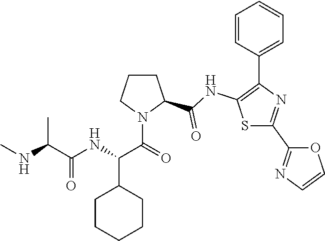 [SMILES: CN[C@@H](C)C(=O)N[C@H](C(=O)N1CCC[C@H]1C(=O)Nc1sc(-c2ncco2)nc1-c1ccccc1)C1CCCCC1]

CUDC 427

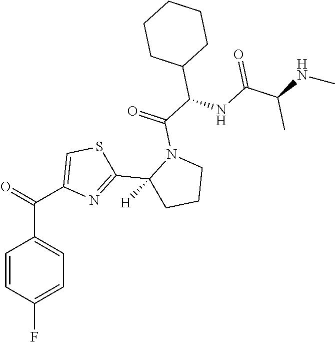 [SMILES: CN[C@@H](C)C(=O)N[C@H](C(=O)N1CCC[C@H]1c1nc(C(=O)c2ccc(F)cc2)cs1)C1CCCCC1]

LCL 161

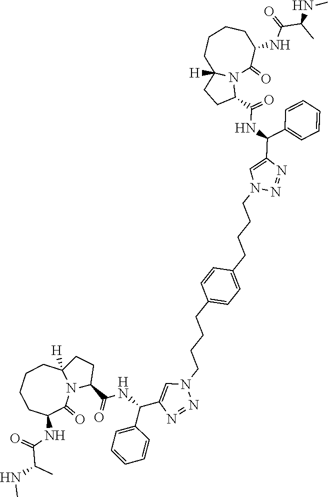 [SMILES: CN[C@@H](C)C(=O)N[C@H]1CCCC[C@H]2CC[C@@H](C(=O)N[C@@H](c3ccccc3)c3cn(CCCCc4ccc(CCCCn5cc([C@@H](NC(=O)[C@@H]6CC[C@@H]7CCCC[C@H](NC(=O)[C@H](C)NC)C(=O)N76)c6ccccc6)nn5)cc4)nn3)N2C1=O]

SM-164

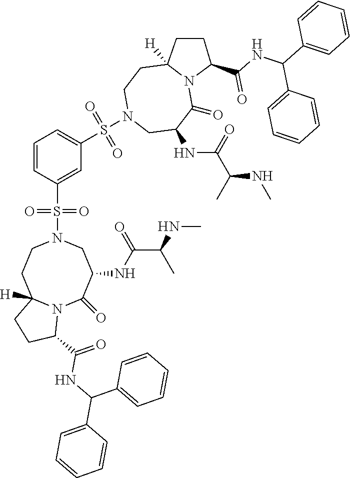 [SMILES: CN[C@@H](C)C(=O)N[C@H]1CN(S(=O)(=O)c2cccc(S(=O)(=O)N3CC[C@H]4CC[C@@H](C(=O)NC(c5ccccc5)c5ccccc5)N4C(=O)[C@@H](NC(=O)[C@H](C)NC)C3)c2)CC[C@H]2CC[C@@H](C(=O)NC(c3ccccc3)c3ccccc3)N2C1=O]

APG-1387

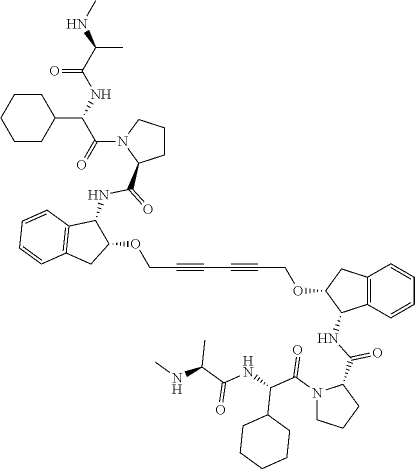 [SMILES: CN[C@@H](C)C(=O)N[C@H](C(=O)N1CCC[C@H]1C(=O)N[C@H]1c2ccccc2C[C@H]1OCC#CC#CCO[C@@H]1Cc2ccccc2[C@@H]1NC(=O)[C@@H]1CCCN1C(=O)[C@@H](NC(=O)[C@H](C)NC)C1CCCCC1)C1CCCCC1]

AZD-5582

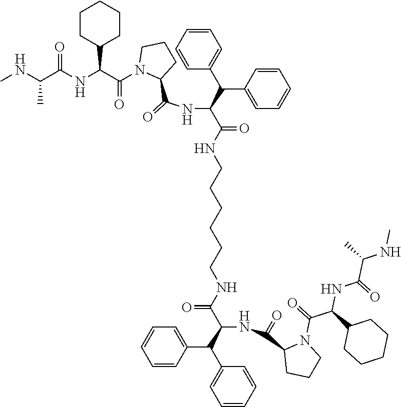

BV6

### Example 2: Treatment of Pulmonary Disease

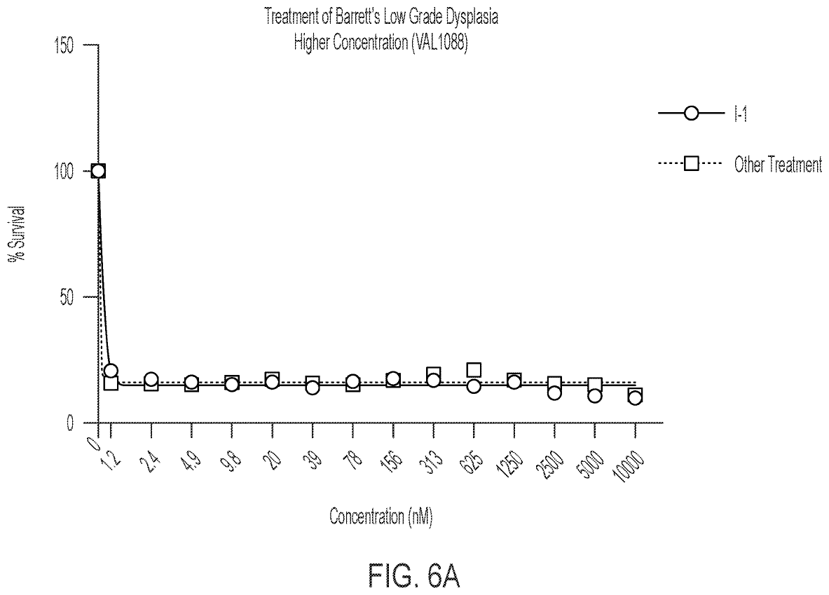

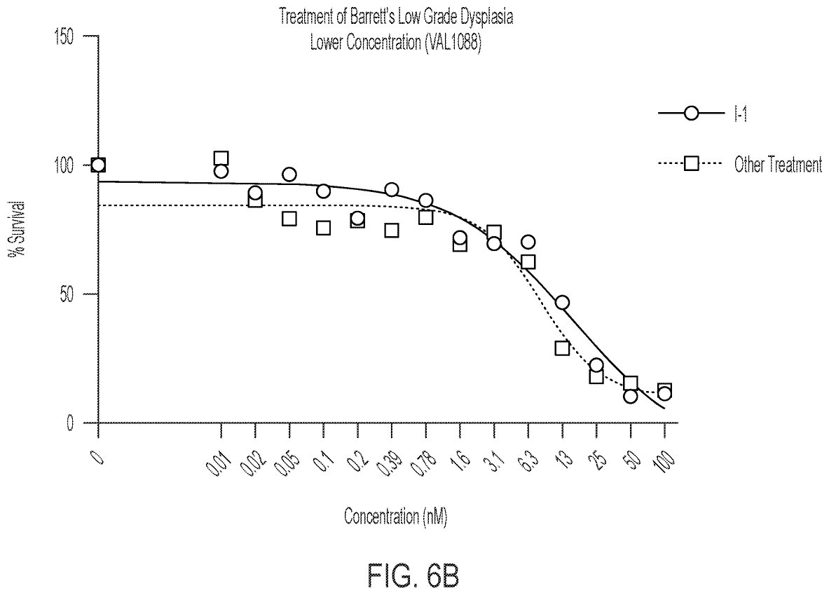

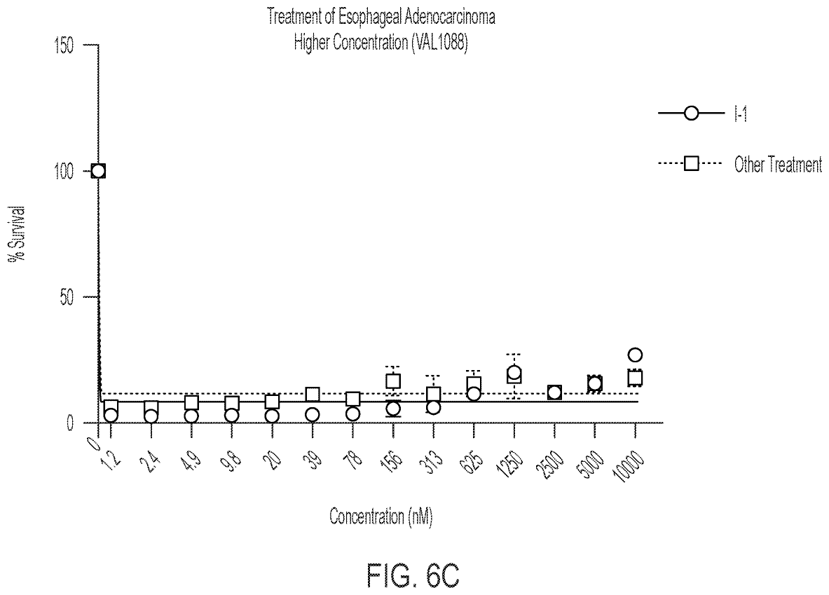

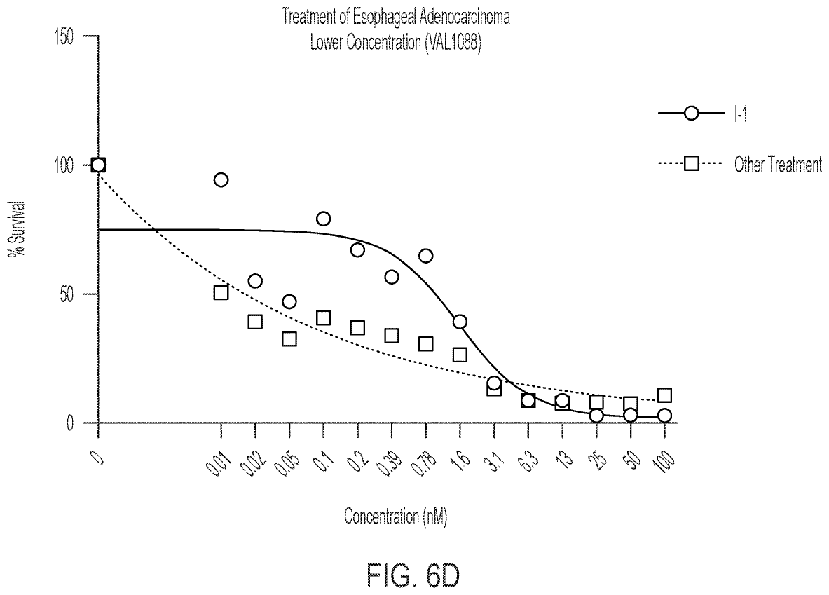

The present example further demonstrates the efficacy of compounds described herein in the treatment of pulmonary diseases. Specifically, esophageal adenocarcinoma stem cells isolated from multiple distinct individuals were treated with compounds described herein. FIG. 2 demonstrates a comparison of compounds described herein (e.g., I-1 and I-2) with other compounds (e.g., IAP inhibitors as listed in Table 1). Notably esophageal adenocarcinoma stem cells were significantly more sensitive to compounds I-1 and I-2 compared to the other compounds as listed in Table 1. Esophageal adenocarcinoma stem cells from an additional individual were tested and treated at higher (nM) or low (pM) concentrations of I-1 (FIG. 6C and FIG. 6D) further demonstrating the sensitivity of pulmonary diseases to the compounds described herein.

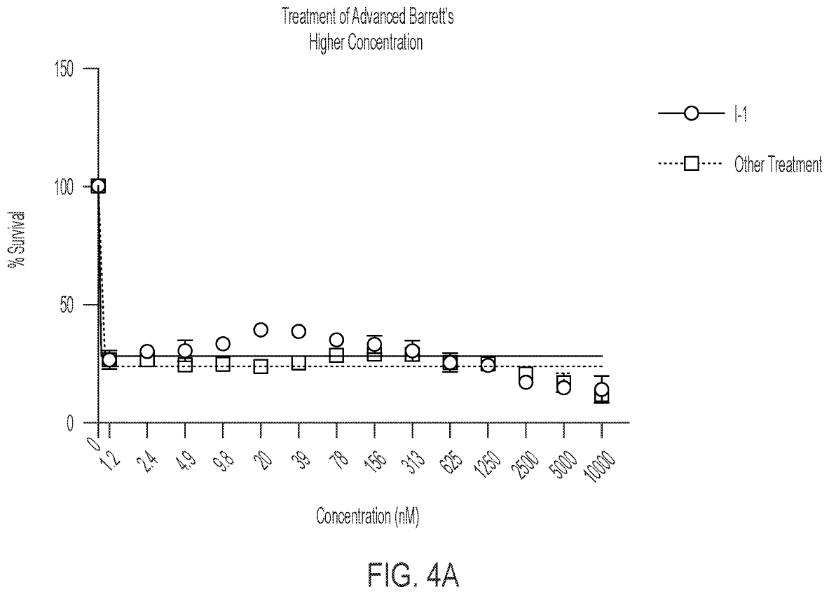

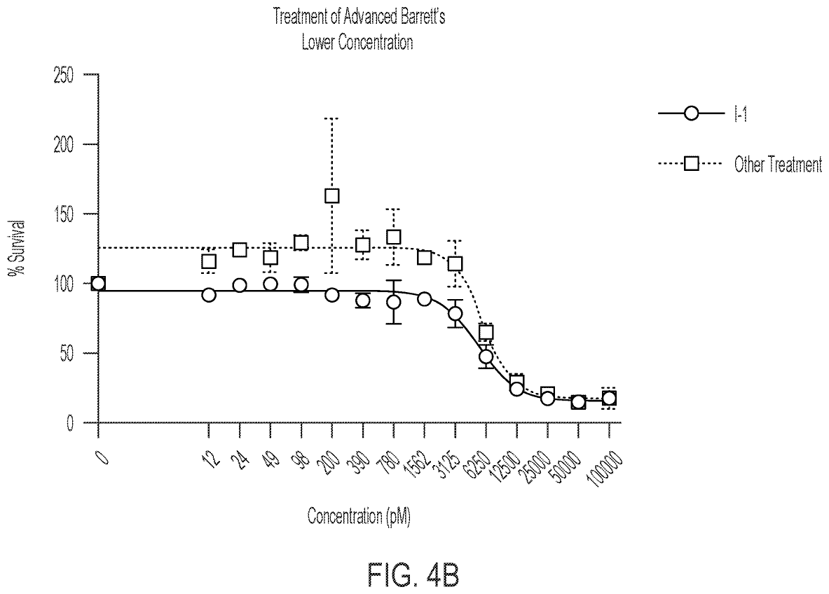

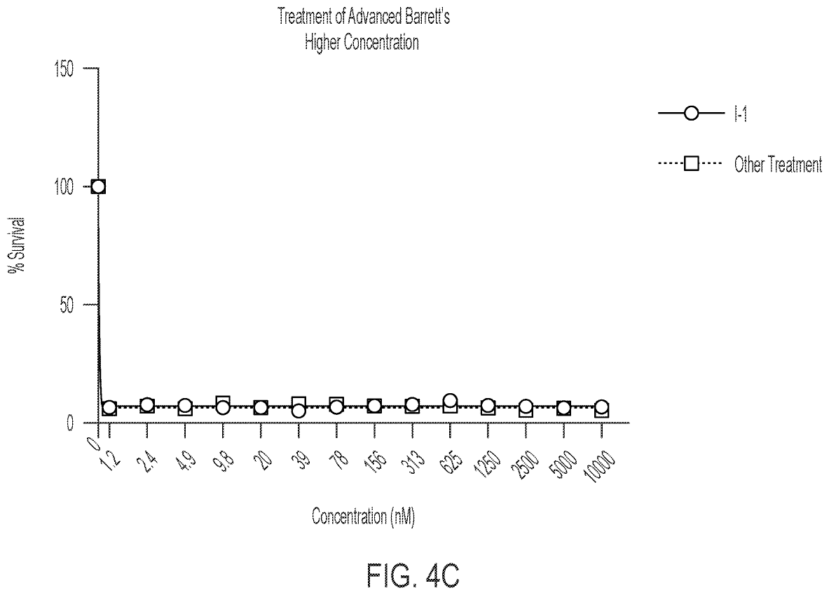

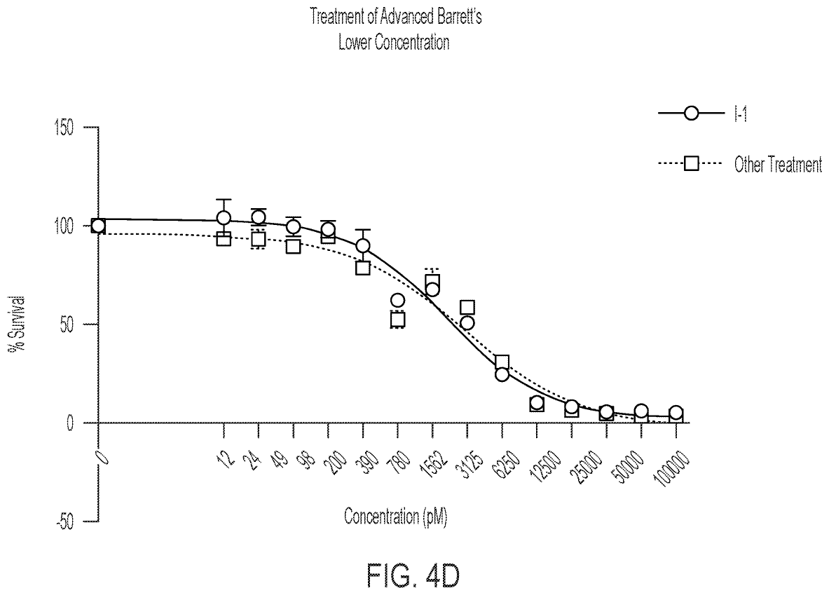

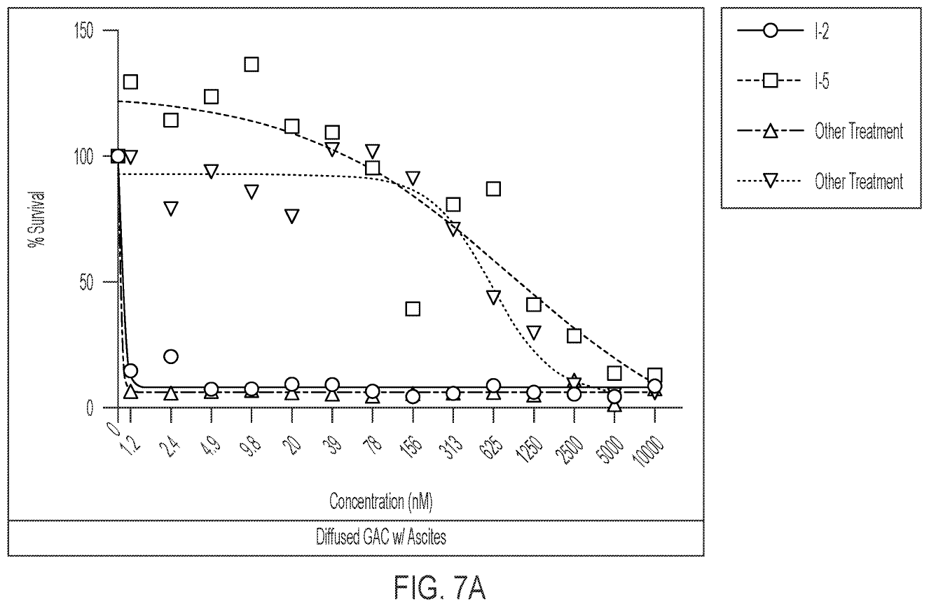

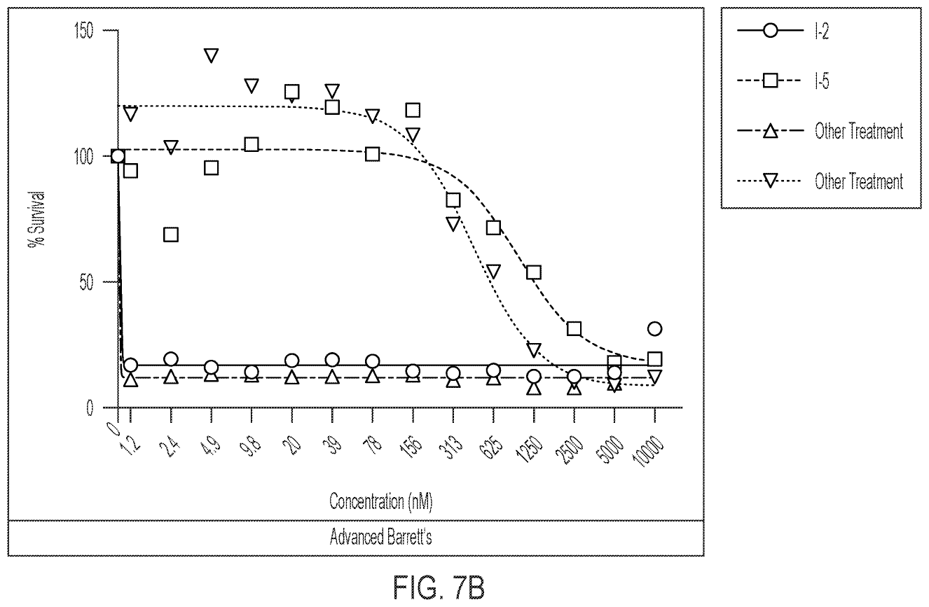

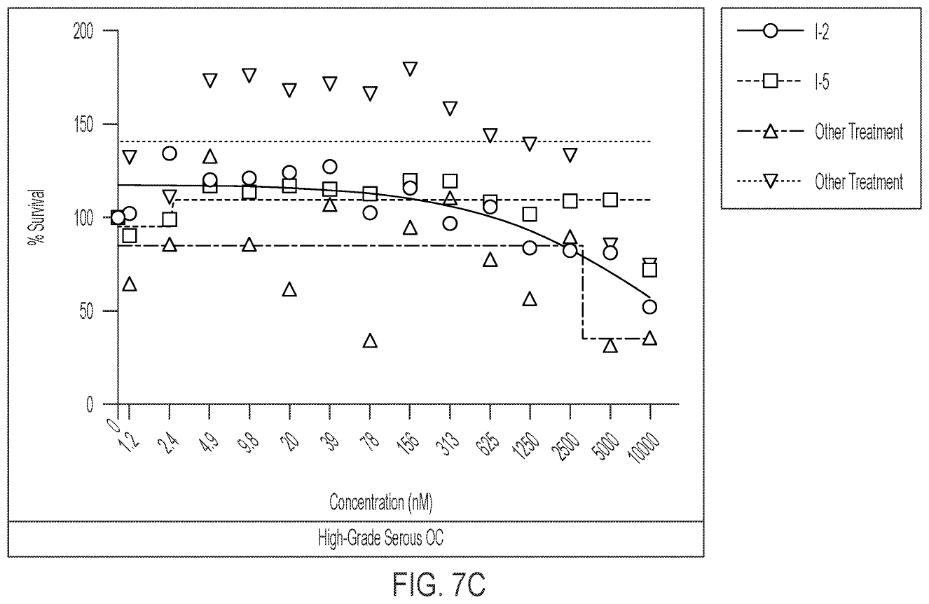

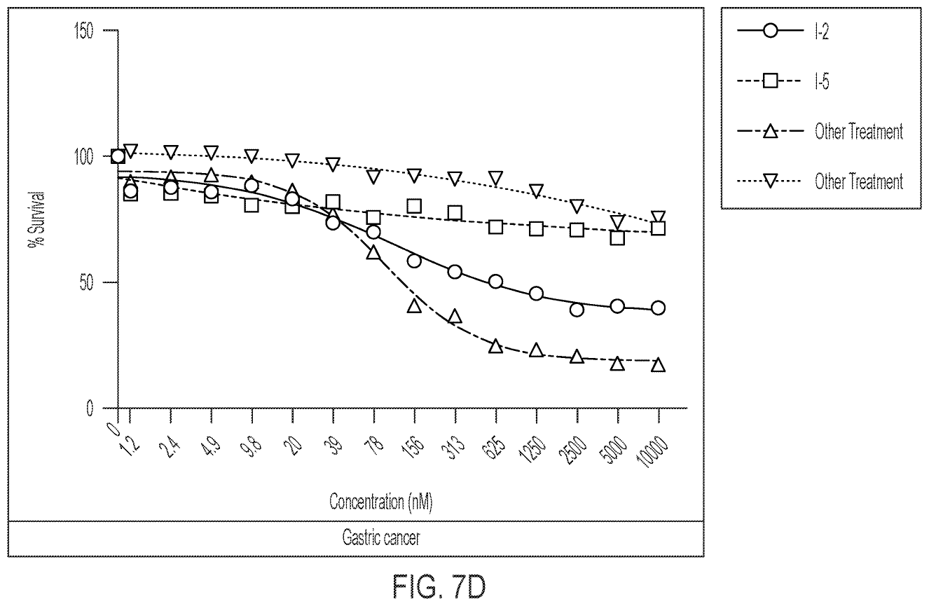

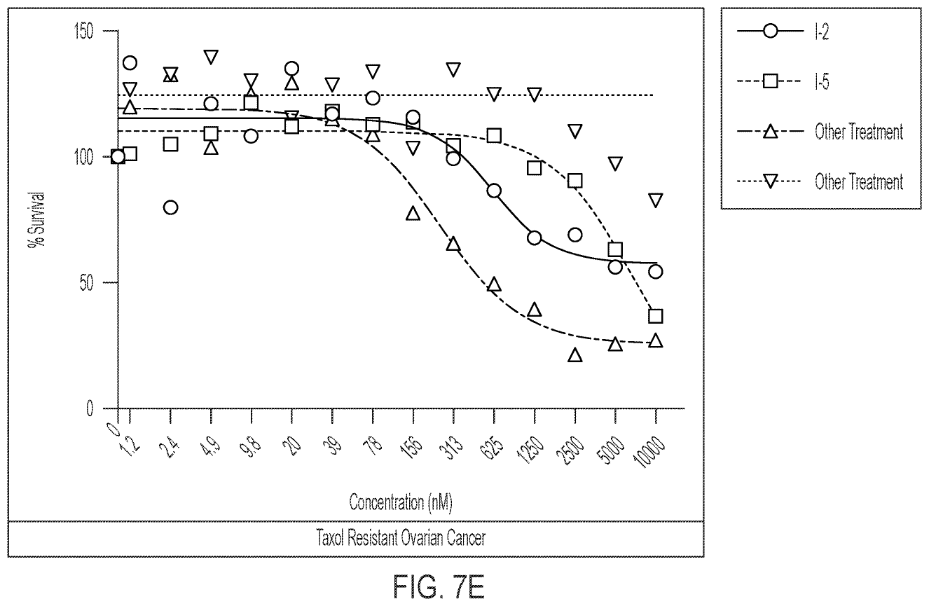

Further, various concentrations of compounds as described herein were tested on isolated advanced Barrett's esophagus stem cells. Isolated advanced Barrett's esophagus stem cells from one individual were treated at higher (nM) or low (pM) concentrations of I-1 (FIGS. 4A and 4B respectively). Similar treatment of advanced Barrett's esophagus stem cells from a different individual also demonstrates the sensitivity of pulmonary diseases to compounds as described herein (e.g., I-1) see FIGS. 4C and 4D. FIG. 7B further demonstrates this sensitivity. Isolated advanced Barrett's esophagus stem cells from an additional individual are notably sensitive to compound I-2 as described herein but not compound I-5.

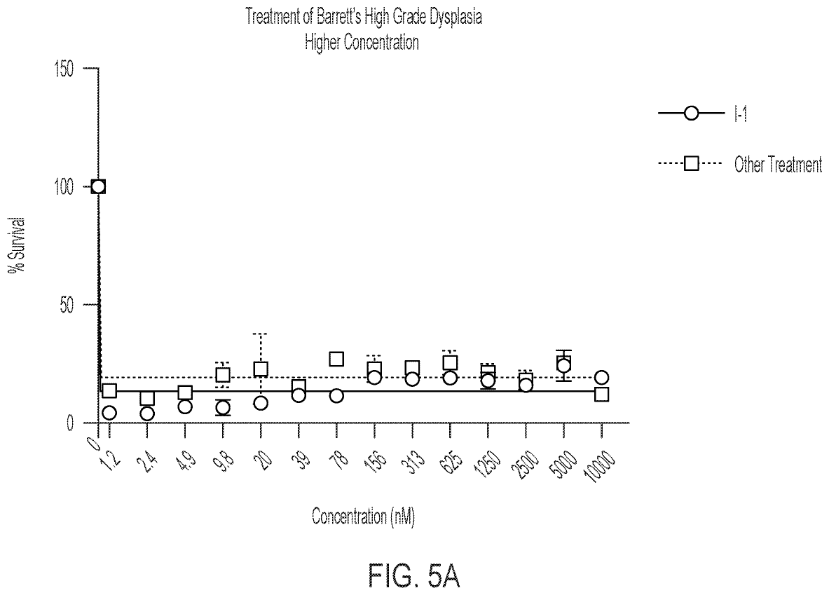

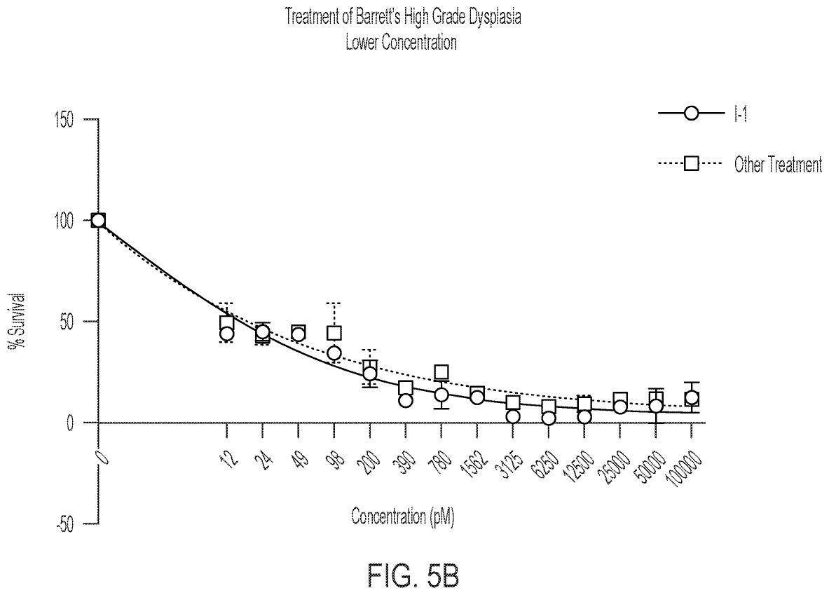

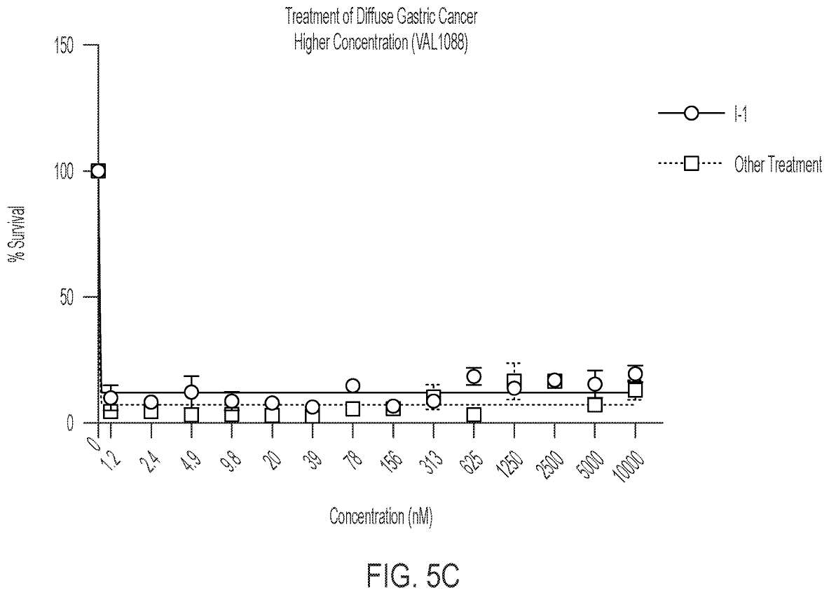

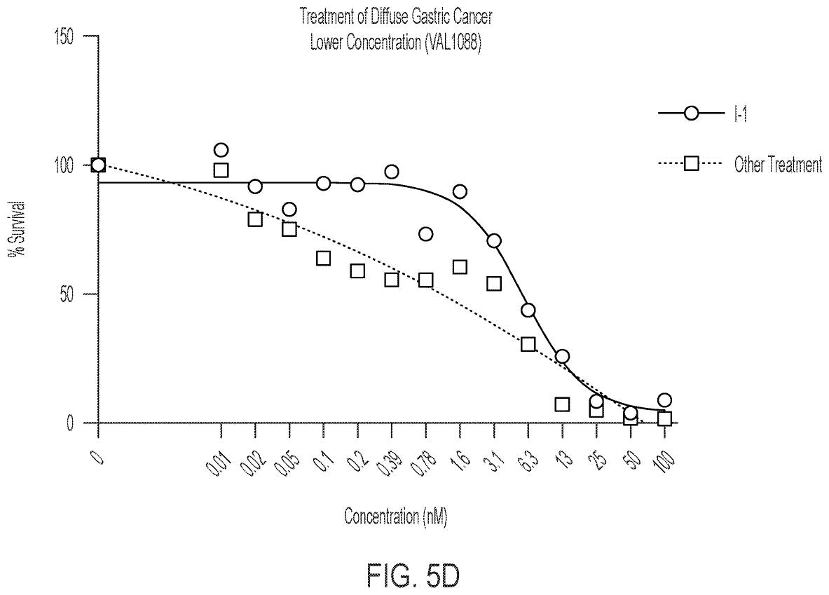

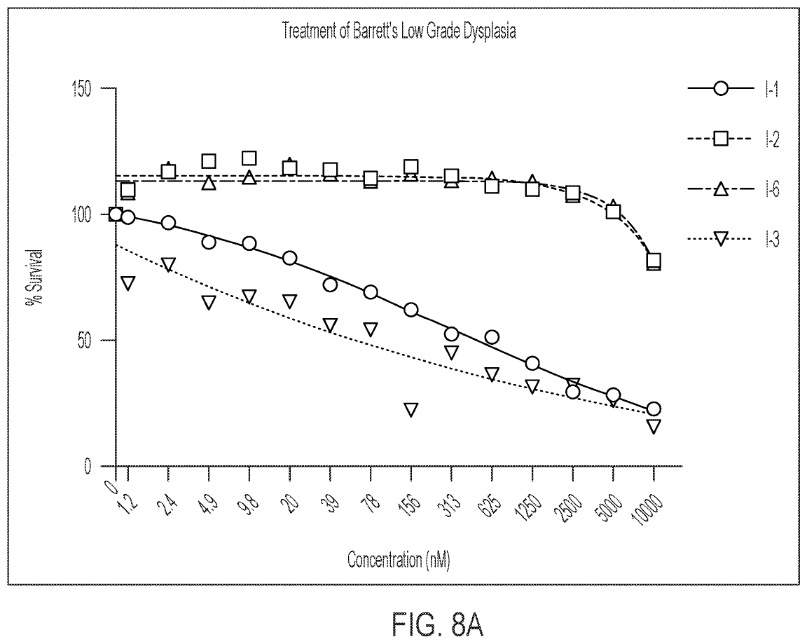

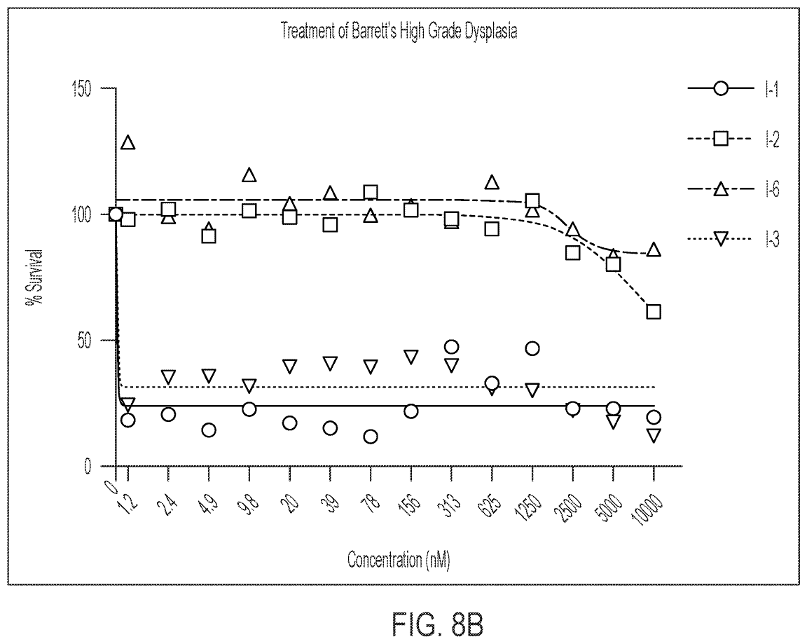

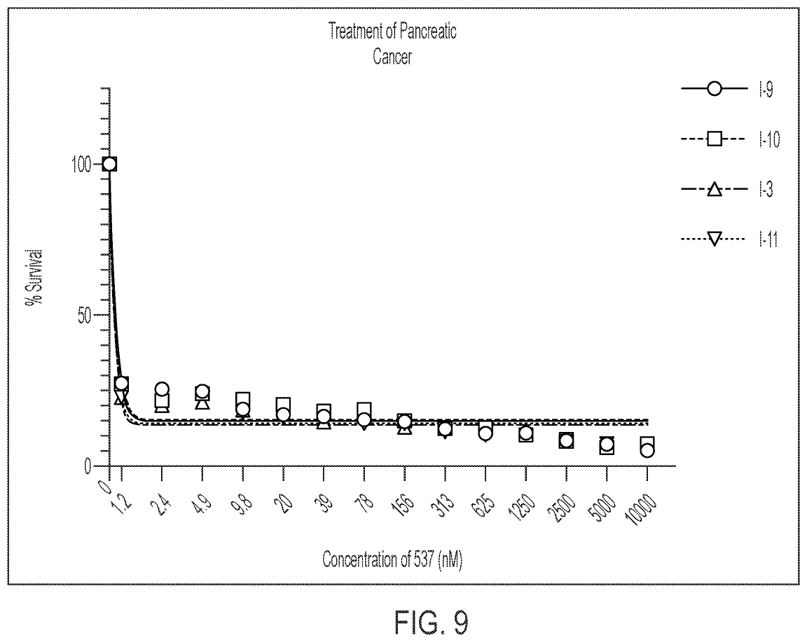

Additionally, various concentrations of compounds as described herein were tested on isolated Barrett's dysplasia stem cells of various grades (i.e., high and low). FIGS. 5A and 5B demonstrates Barrett's high grade dysplasia stem cells are sensitive to I-1. FIGS. 6A and 6B demonstrates Barrett's low grade dysplasia stem cells are sensitive to I-1. Compounds I-1, I-2, I-6, and I-3 were tested at various concentrations on Barrett's low grade dysplasia stem cells from one individual (FIG. 8A) and Barrett's high grade dysplasia stem cells from another individual (FIG. 8B).

### Example 3: Treatment of Cancers

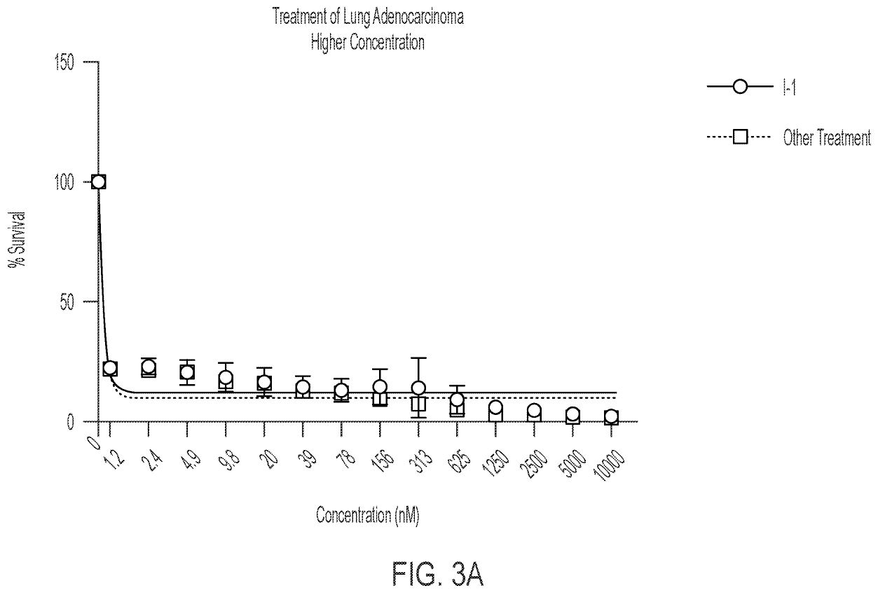

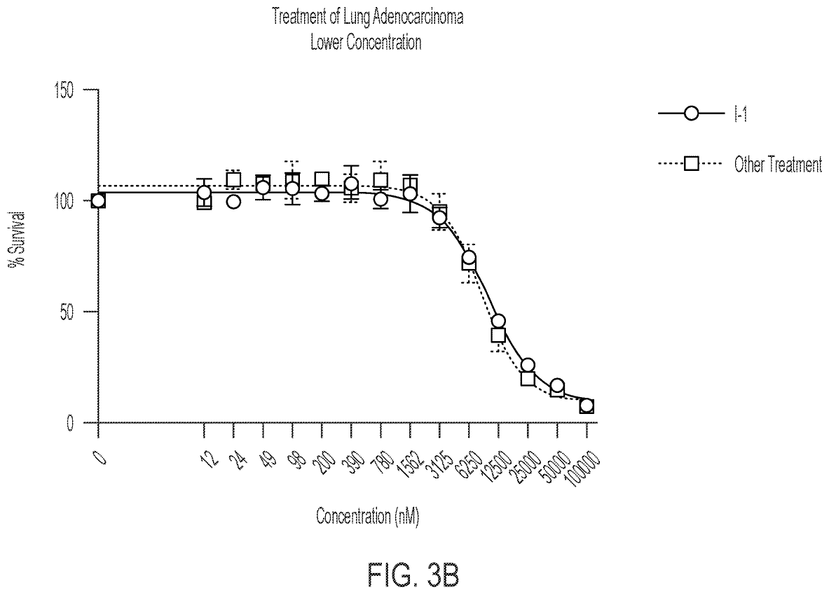

In addition to the cancers described above (e.g., esophageal adenocarcinoma) the present example further demonstrates the sensitivity of various cancers to the compounds described herein. Specifically, lung adenocarcinoma stem cells from an additional individual were tested and treated at higher (nM) or low (pM) concentrations of I-1 (FIG. 3A and FIG. 3B). These results demonstrate the sensitivity of cancers to the compounds as described herein.

Further, stem cells isolated from an individual suffering from diffuse gastric cancer were tested and treated at higher or low concentrations of I-1 (FIG. 5C and FIG. 5D). Stem cells isolated from two additional individuals suffering from diffuse gastric cancer were tested and treated with compounds I—2 and I-5 (FIGS. 7A and 7D). Notably, gastric cancer stem cells were sensitive to I-2 as described herein but not compound I-5.

As a further demonstration of the breadth of diseases sensitive to the componds as described herein pancreatic cancer stem cells were isolated and treated with compounds I-9, I-10, I-3, and I-11. FIG. 7C demonstrates sensitivity of high grade serous ovarian cancer stem cells to compounds described herein. Moreover, FIG. 7E demonstrates sensitivity of taxol resistant ovarian cancer stem cells to compounds described herein.

### Example 4: Compounds Described Herein are Non-Toxic

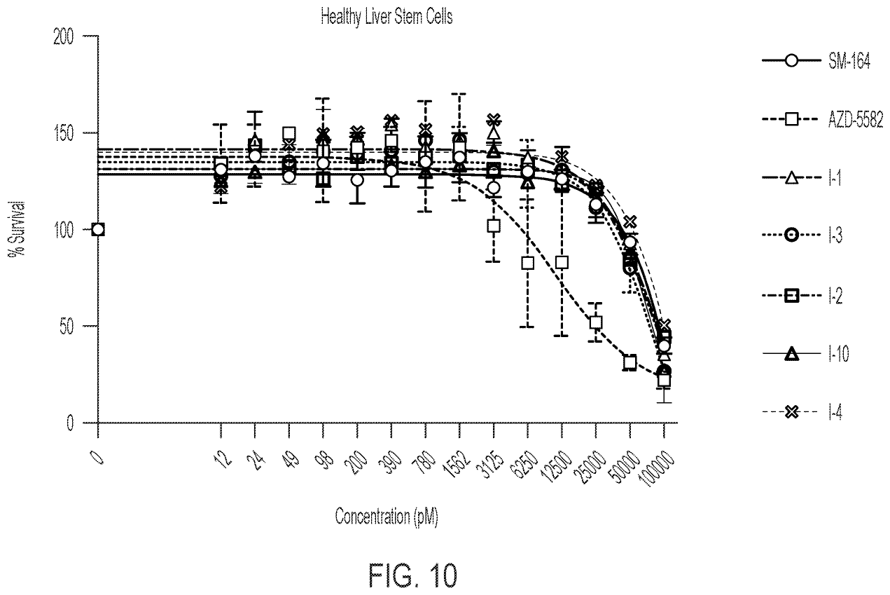

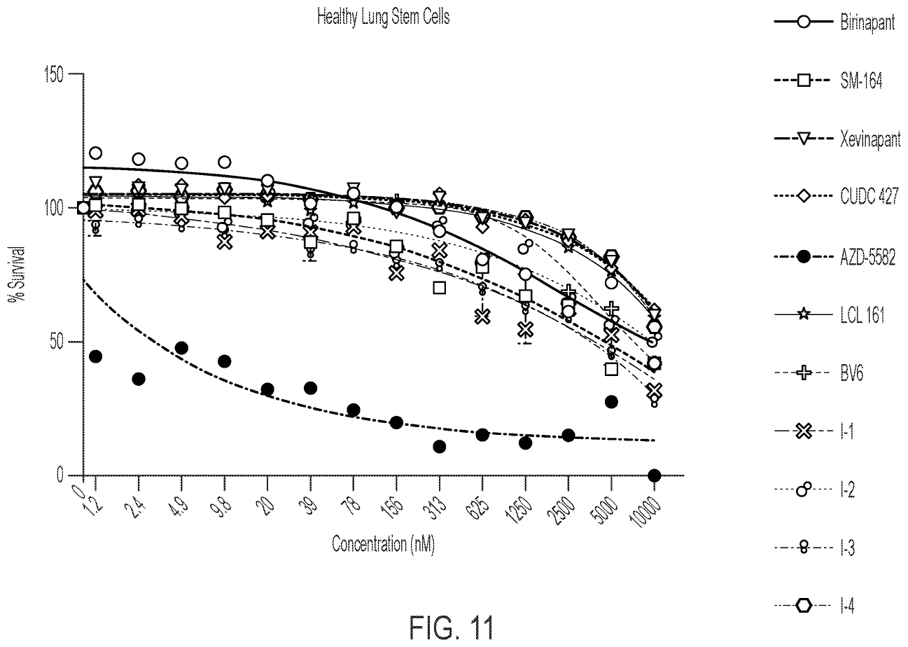

The present example demonstrates that the compounds described herein are not toxic to healthy cells. Healthy liver cells were treated with various known compounds as well as compounds as described in the present disclosure (FIG. 10). Additionally, healthy lung cells were treated with various known compounds as well as compounds as described in the present disclosure (FIG. 11). The present compounds did not demonstrate toxicity except at higher concentrations.

### Example 5: Methods for Testing Efficacy of Compounds Described Herein

The present example describes methods used for testing the efficacy of compounds described herein. The selective growth of stem cells of cancers, of their precursor lesions, and of normal and chronically diseased epithelial tissues is described in the references below. In brief, biopsied tissues are reduced to single cell suspensions and plated onto lawns of irradiated 3T3-J2 feeder cells in specialized StemEcho media and libraries of colonies are evident in 7-10 days. Single cell-derived clones are generated by single cell FACS sorting to 384 well plates and wells with individual colonies are expanded, analyzed by molecular genetics, and grown as discrete clones.

Approximately 400,000 cells from discrete clones relevant to particular disease states (cancer, chronic inflammatory disease) are plated into 384 plates previously seeded with irradiated feeder cells, allowed to grow for 5 days, and then exposed to the test compounds in a serial dilution format. After 2-5 days, the cells are fixed using paraformaldehyde and the human cells are labelled with human-specific antibodies followed by fluorochrome-labelled secondary antibodies. Human cell numbers are quantified via high-throughput imaging technology (CellInsight CX7 LED, Thermo), and data analyzed by Excel.

### REFERENCES

Wang X, Yamamoto Y, Wilson L H, Zhang T, Howitt B, Farrow M A, Kern F, Ning G, Yue Hong, Khor C C, Chevalier B, Bertrand D, Nagarajan N, Sylvester F A, Hyams J S, Devers T, Bronson R, Lacy D B, Ho K Y, Crum C P, McKeon F and Xian W. (2015). Cloning and variation of ground state intestinal stem cells. Nature 522, 173-178.
Yamamoto Y, Wang X, Bertrand D, Kern F, Zhang T, Hu Y Y, Deluba M, Srivastava S, Ming T, Khor C C, Wilson L, Blaszyk H, Rolshud D, Liu J J, Howitt B, Crum C P, Nagarajan N, Ho K Y, McKeon F, and Xian W. 2016. Mutational Spectrum of Barrett's Stem Cells Suggests Paths to Initiation and Progression of a Precancerous Lesion. Nat Commun. 2016 Jan. 19; 7:10380.
Qi Y, Mahalingam R, Flynn K, Rinaldi F, Liew A A, Neupane R, Vincent M, Crum C P, Ho K Y, Hou J K, Hyams J S, Sylvester F A, McKeon F, and Xian W. (2019) An Efficient Method for Cloning Gastrointestinal Stem Cells from Patients via Endoscopic Biopsies. Gastroenterol. 156 (1):20-23.
Duleba M, Yamamoto Y, Neupane R, Rao W, Xie J Z, Qi Y, Liew A A, Niroula S, Zhang Y T, Mahalingam R, Wang S, Goller K, Ajani J A, Vincent M, Ho K K, Hou J K, Hyams J S, Sylvester F A, Crum C P, McKeon F, and Xian W. (2019). Cloning of Ground State Intestinal Stem Cells from Endoscopic Biopsies. Nature Protocol. 15, 1612-1627.
W. Rao, S. Niroula, S. Wang, M. Vincent, F. McKeon, W. Xian, Protocol for Cloning Epithelial Stem Cell Variants from Human Lung. STAR Protoc 1 (2020).
Rao W, Wang S, Duleba M, Niroula S, Goller K, Xie J, et al. Regenerative metaplastic clones in COPD lung drive inflammation and fibrosis. Cell 2020, 181, 848-864.e818.
Wang S, Rao W, Hoffman A, Lin J, L1 J, Lin T, et al. Cloning a profibrotic stem cell variant in idiopathic pulmonary fibrosis. Sci Transl Med. 2023 15(693):eabp9528.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than the specific embodiments that have been represented by way of examples.

1. A compound of formula I: 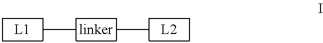
or a pharmaceutically acceptable salt thereof,
wherein:
L1 is a first ligand;
L2 is a second ligand; and
linker is a bivalent linker comprising

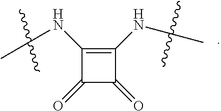 [SMILES: *Nc1c(N*)c(=O)c1=O]

2. The compound of claim 1, wherein each of L1 and L2 is independently a moiety that binds to one or more Inhibitor of Apoptosis Proteins (IAPs).

3. The compound of claim 2, wherein an IAP is selected from Cp-IAP, Op-IAP, XIAP, cIAP1, C-IAP2, NAIP, Livin, or Survivin.

4. The compound of any one of claims 1-3, wherein each of L1 and L2 independently comprises a group selected from: 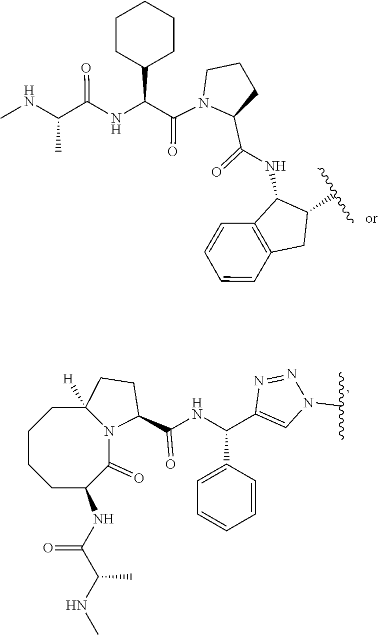 [SMILES: *[C@@H]1Cc2ccccc2[C@@H]1NC(=O)[C@@H]1CCCN1C(=O)[C@@H](NC(=O)[C@H](C)NC)C1CCCCC1 ; *n1cc([C@@H](NC(=O)[C@@H]2CC[C@@H]3CCCC[C@H](NC(=O)[C@H](C)NC)C(=O)N32)c2ccccc2)nn1]
or a pharmaceutically acceptable salt thereof.

5. The compound of any one of claims 1-4, wherein L1 is: 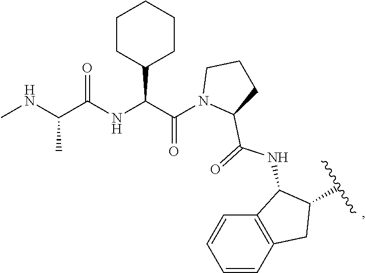 [SMILES: *[C@@H]1Cc2ccccc2[C@@H]1NC(=O)[C@@H]1CCCN1C(=O)[C@@H](NC(=O)[C@H](C)NC)C1CCCCC1]
or a pharmaceutically acceptable salt thereof.

6. The compound of any one of claims 1-4, wherein L1 is: 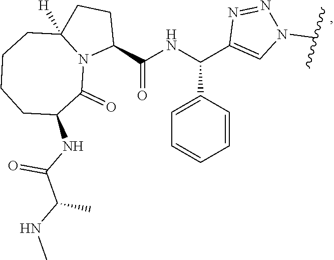 [SMILES: *n1cc([C@@H](NC(=O)[C@@H]2CC[C@@H]3CCCC[C@H](NC(=O)[C@H](C)NC)C(=O)N32)c2ccccc2)nn1]
or a pharmaceutically acceptable salt thereof.

7. The compound of any one of claims 1-6, wherein L2 is: 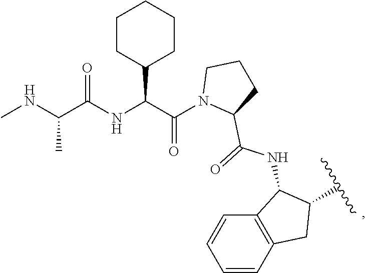 [SMILES: *[C@@H]1Cc2ccccc2[C@@H]1NC(=O)[C@@H]1CCCN1C(=O)[C@@H](NC(=O)[C@H](C)NC)C1CCCCC1]
or a pharmaceutically acceptable salt thereof.

8. The compound of any one of claims 1-6, wherein L2 is: 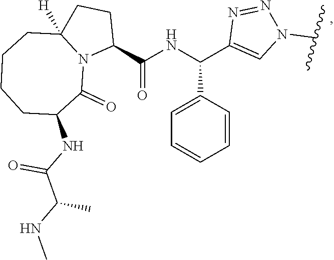 [SMILES: *n1cc([C@@H](NC(=O)[C@@H]2CC[C@@H]3CCCC[C@H](NC(=O)[C@H](C)NC)C(=O)N32)c2ccccc2)nn1]
or a pharmaceutically acceptable salt thereof.

9. The compound of any one of claims 1-3, wherein the compound is of formula I-a, I-b, or I-c: 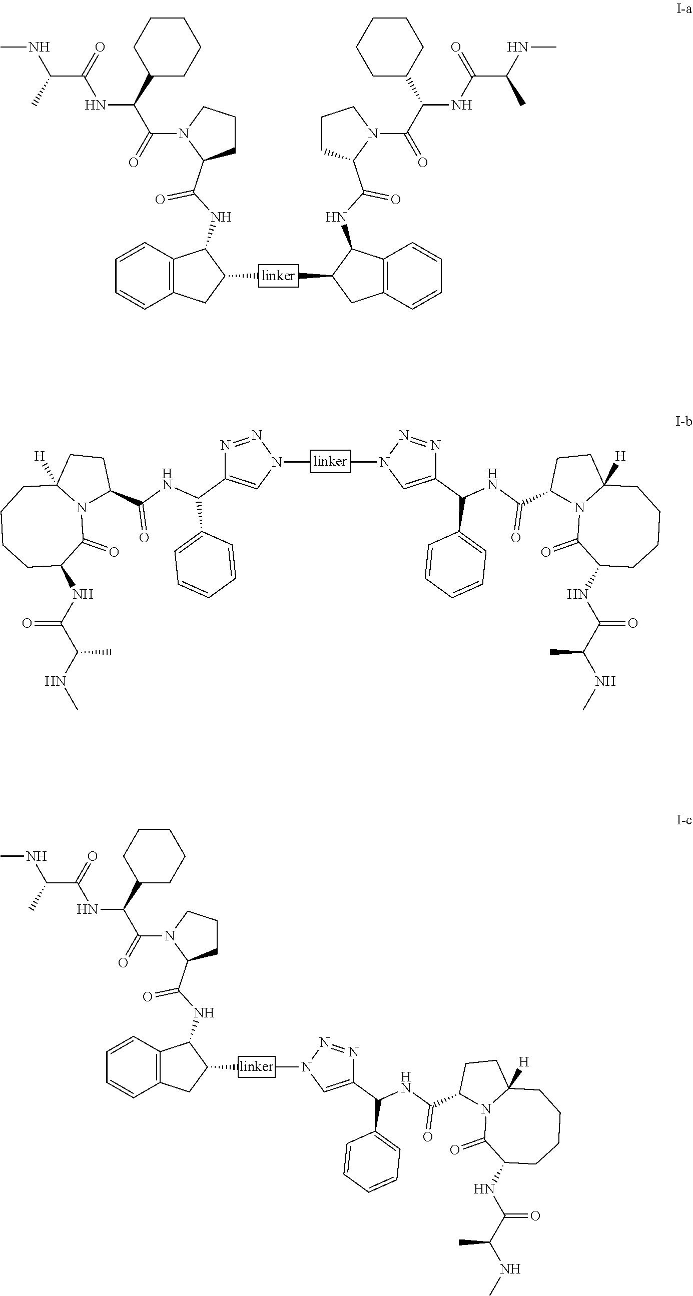
or a pharmaceutically acceptable salt thereof.

10. The compound of any one of claims 1-9, wherein the linker is of formula X: 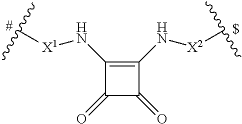
or a pharmaceutically acceptable salt thereof, wherein:
each of X¹ and X² is independently a covalent bond or an optionally substituted bivalent, saturated or partially unsaturated, straight or branched C₁₋₁₂ hydrocarbon chain, wherein 1-4 carbon atoms are optionally and independently replaced by —O—, —N(R)—, —C(O)—, —S—, —SO—, —SO₂—, or -Cy-;
each R is independently selected from hydrogen or an optionally substituted C₁₋₆ aliphatic;
each -Cy- is independently an optionally substituted bivalent ring selected from a 3- to 8-membered carbocyclene, a 5- to 6-membered saturated or partially unsaturated heterocyclene having 1-3 heteroatoms independently selected from oxygen, nitrogen, or sulfur; phenylene; or a 5- to 6-membered heteroarylene having 1-3 heteroatoms independently selected from oxygen, nitrogen, or sulfur;
#represents the point of attachment to L1; and
$ represents the point of attachment to L2.

11. The compound of claim 10, wherein X¹ and X² are the same.

12. The compound of claim 10, wherein X¹ and X² are different.

13. The compound of claim 10, wherein each of X¹ and X² is independently a covalent bond or an optionally substituted bivalent, saturated or partially unsaturated, straight or branched C₁₋₆ hydrocarbon chain, wherein 1-2 carbon atoms are optionally and independently replaced by —O—, —N(R)—, or —C(O)—.

14. The compound of claim 10 or 13, wherein X¹ is a covalent bond.

15. The compound of claim 10 or 13, wherein X¹ an optionally substituted bivalent, saturated or partially unsaturated, straight C₃₋₆ hydrocarbon chain, wherein 1-2 carbon atoms are optionally replaced by —O—.

16. The compound of any one of claims 10, 13, or 15, wherein X¹ is an optionally substituted bivalent, saturated, straight C₃ hydrocarbon chain, wherein 1 carbon atom is replaced by —O—.

17. The compound of any one of claims 10, 13, or 15, wherein X¹ is an optionally substituted bivalent, saturated, straight C₄ hydrocarbon chain.

18. The compound of any one of claims 10, 13, or 15, wherein X¹ is an optionally substituted bivalent, saturated, straight C₅ hydrocarbon chain, wherein 1 carbon atom is replaced by —O—.

19. The compound of any one of claims 10, 13, or 15, wherein X¹ is an optionally substituted bivalent, saturated, straight C₅ hydrocarbon chain.

20. The compound of any one of claims 10, 13, or 15, wherein X¹ is an optionally substitute bivalent, saturated, straight C₆ hydrocarbon chain, wherein 1 carbon atom is replaced by —O—.

21. The compound of any one of claims 10, 13, or 15, wherein X¹ is an optionally substitute bivalent, saturated, straight C₆ hydrocarbon chain, wherein 2 carbon atoms are replaced by —O—.

22. The compound of any one of claims 10, 13, or 15, wherein X¹ is:
covalent bond, 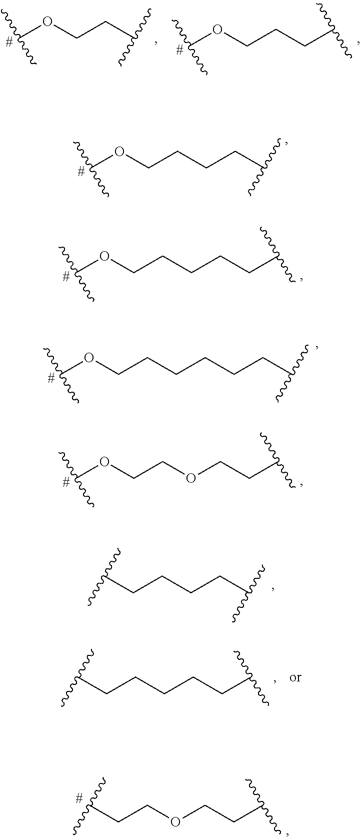
wherein #represents the point of attachment to L1.

23. The compound of any one of claims 10 or 13-22, wherein X² is a covalent bond.

24. The compound of any one of claims 10 or 13-22, wherein X² an optionally substituted bivalent, saturated or partially unsaturated, straight C₃₋₆ hydrocarbon chain, wherein 1-2 carbon atoms are optionally replaced by —O—.

25. The compound of any one of claims 10, 13-22, or 24, wherein X² is an optionally substituted bivalent, saturated, straight C₃ hydrocarbon chain, wherein 1 carbon atom is replaced by —O—.

26. The compound of any one of claims 10, 13-22, or 24, wherein X² is an optionally substituted bivalent, saturated, straight C₄ hydrocarbon chain.

27. The compound of any one of claims 10, 13-22, or 24, wherein X² is an optionally substituted bivalent, saturated, straight C₅ hydrocarbon chain, wherein 1 carbon atom is replaced by —O—.

28. The compound of any one of claims 10, 13-22, or 24, wherein X² is an optionally substituted bivalent, saturated, straight C₅ hydrocarbon chain.

29. The compound of any one of claims 10, 13-22, or 24, wherein X² is an optionally substitute bivalent, saturated, straight C₆ hydrocarbon chain, wherein 1 carbon atom is replaced by —O—.

30. The compound of any one of claims 10, 13-22, or 24, wherein X² is an optionally substitute bivalent, saturated, straight C₆ hydrocarbon chain, wherein 2 carbon atoms are replaced by —O—.

31. The compound of any one of claims 10, 13-22, or 24, wherein X² is:
covalent bond, 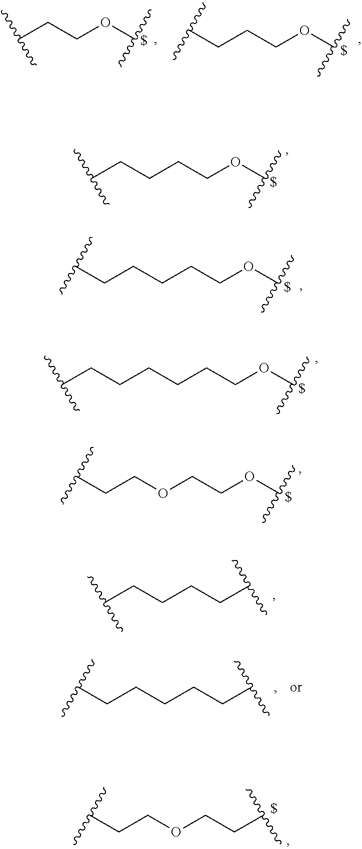
wherein $ represents the point of attachment to L2.

32. The compound of any one of claims 1-31, wherein the compound is of formula I-a 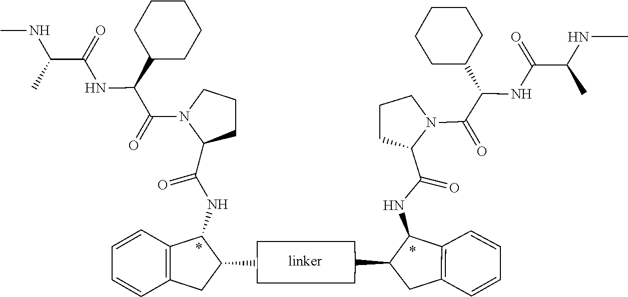
or a pharmaceutically acceptable salt thereof, wherein
the linker is sufficient to position L1 and L2 at a distance of about 0.7-2.2 nm between the C1 carbon atoms of the respective indanyl groups indicated by * below:

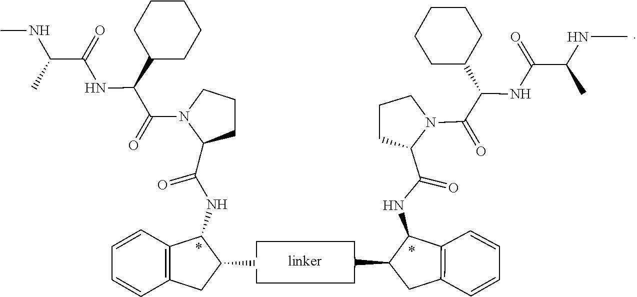

33. The compound of claim 32, wherein the linker is sufficient to position L1 and L2 at a distance of about 0.7-0.8, 1.4-1.5, or 2.0-2.2 nm between the C1 carbon atoms of the respective indanyl groups.

34. The compound of any one of claims 1-31, wherein the compound is of formula I-b: 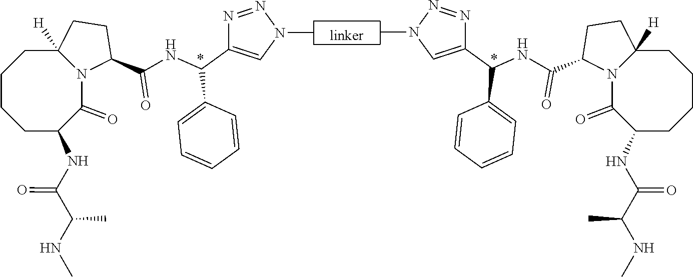
or a pharmaceutically acceptable salt thereof, wherein
the linker is sufficient to position L1 and L2 at a distance of about 1.9-2.2 nm between the respective benzylic carbon atoms (indicated by * below):

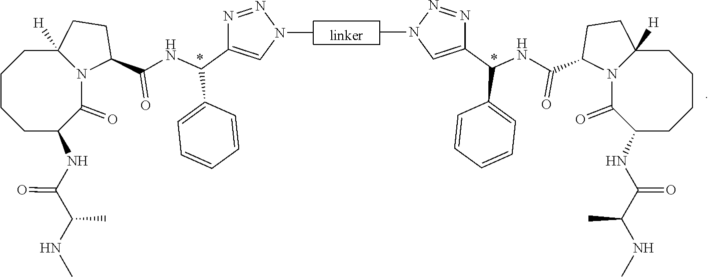

35. The compound of any one of claims 1-31, wherein the compound is of formula I-c: 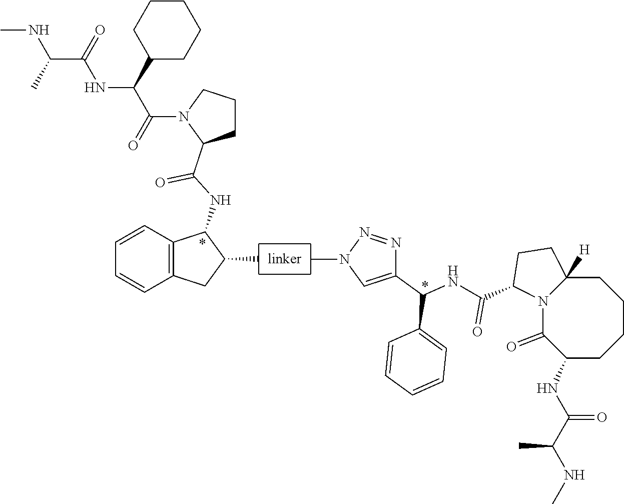
or a pharmaceutically acceptable salt thereof, wherein
the linker is sufficient to position L1 and L2 at a distance of about 1.9-2.1 nm between the indanyl carbon atom of L1 and the benzylic carbon atom of L2 (indicated by * below):

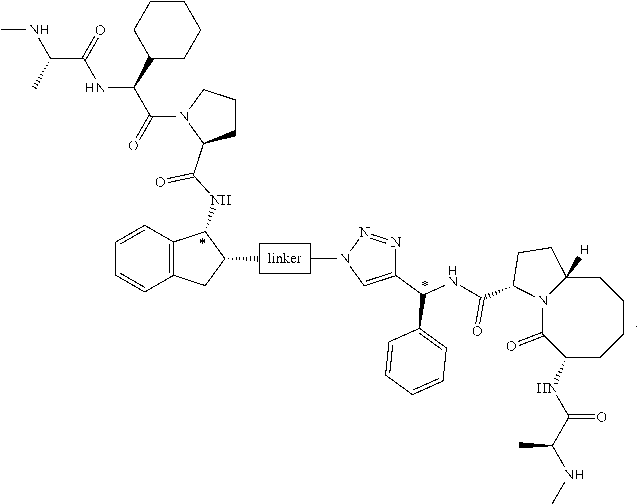

36. The compound of claim 1, wherein the compound is selected from:
Compound

| | |
|---|---|
| 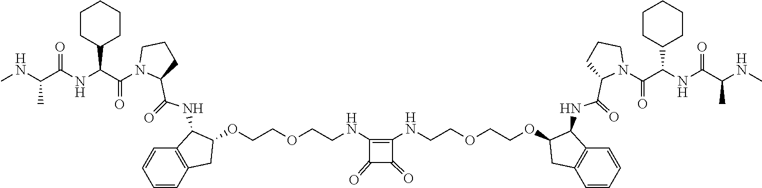 | I-1 |
| 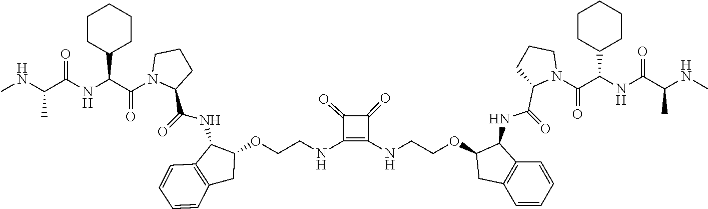 | I-2 |
| 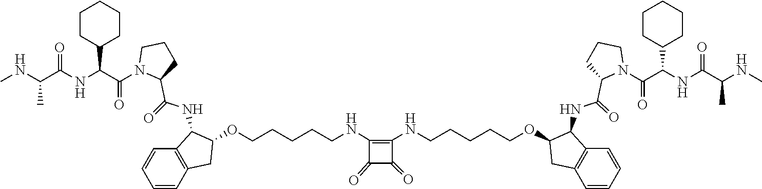 | I-3 |
| 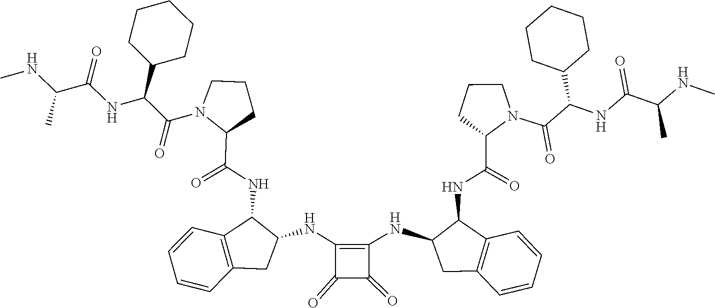 | I-4 |
| 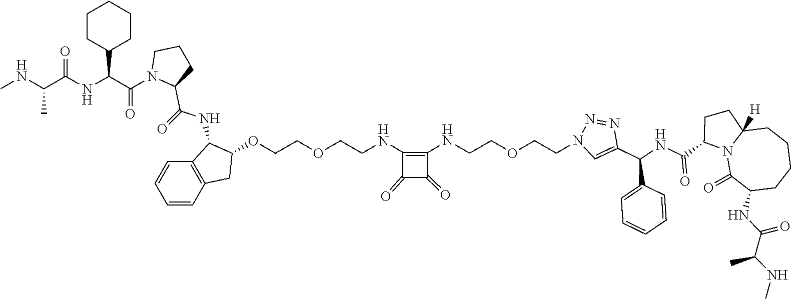 [SMILES: CN[C@@H](C)C(=O)N[C@H]1CCCC[C@H]2CC[C@@H](C(=O)N[C@@H](c3ccccc3)c3cn(CCOCCNc4c(NCCOCCO[C@@H]5Cc6ccccc6[C@@H]5NC(=O)[C@@H]5CCCN5C(=O)[C@@H](NC(=O)[C@H](C)NC)C5CCCCC5)c(=O)c4=O)nn3)N2C1=O] | I-5 |
| 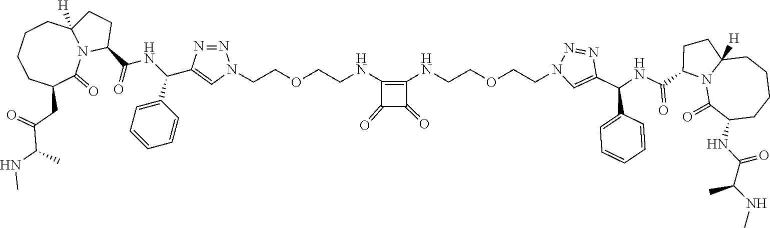 [SMILES: CN[C@@H](C)C(=O)C[C@H]1CCCC[C@H]2CC[C@@H](C(=O)N[C@@H](c3ccccc3)c3cn(CCOCCNc4c(NCCOCCn5cc([C@@H](NC(=O)[C@@H]6CC[C@@H]7CCCC[C@H](NC(=O)[C@H](C)NC)C(=O)N76)c6ccccc6)nn5)c(=O)c4=O)nn3)N2C1=O] | I-6 |
| 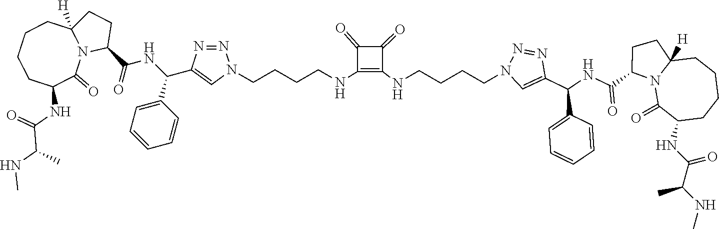 [SMILES: CN[C@@H](C)C(=O)N[C@H]1CCCC[C@H]2CC[C@@H](C(=O)N[C@@H](c3ccccc3)c3cn(CCCCNc4c(NCCCCn5cc([C@@H](NC(=O)[C@@H]6CC[C@@H]7CCCC[C@H](NC(=O)[C@H](C)NC)C(=O)N76)c6ccccc6)nn5)c(=O)c4=O)nn3)N2C1=O] | I-7 |
| 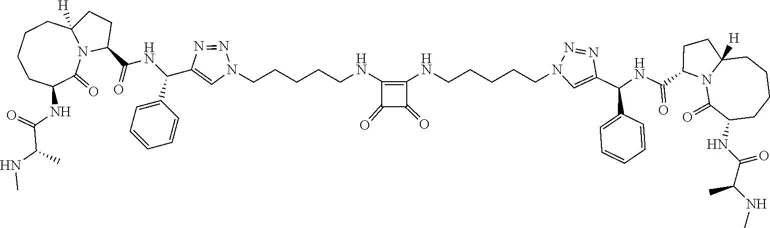 [SMILES: CN[C@@H](C)C(=O)N[C@H]1CCCC[C@H]2CC[C@@H](C(=O)N[C@@H](c3ccccc3)c3cn(CCCCCNc4c(NCCCCCn5cc([C@@H](NC(=O)[C@@H]6CC[C@@H]7CCCC[C@H](NC(=O)[C@H](C)NC)C(=O)N76)c6ccccc6)nn5)c(=O)c4=O)nn3)N2C1=O] | I-8 |
| 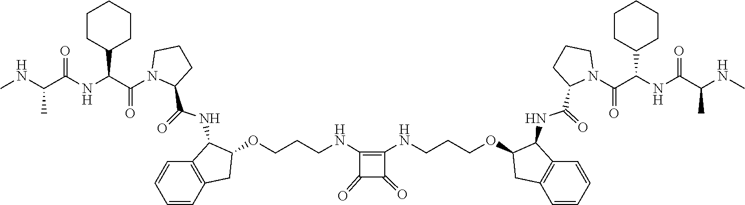 | I-9 |
| 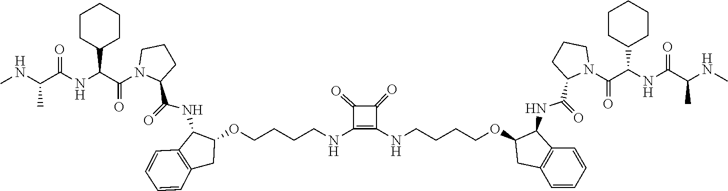 | I-10 |
| 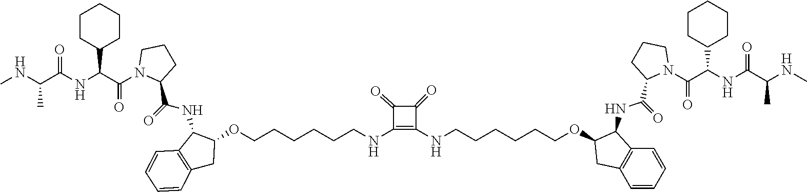 | I-11 |

or a pharmaceutically acceptable salt thereof.

37. A pharmaceutical composition comprising a compound according to any one of claims 1-36, or a pharmaceutically acceptable salt thereof, and a pharmaceutically acceptable carrier, adjuvant, or vehicle.

38. A method of inhibiting activity of one or more IAPs, or variants or mutants thereof, in a biological sample or in a patient, the method comprising a step of contacting the biological sample or administering to a patient a compound according to any one of claims 1-36, or a pharmaceutically acceptable salt thereof.

39. A method of treating a disease or disorder associated with one or more IAPs, the method comprising a step of administering to a patient in need thereof a compound according to any one of claims 1-40, or a pharmaceutically acceptable salt thereof.

40. The method according to claim 39, wherein the disease or disorder associated with one or more IAPs is a cancer.

41. The method according claim 40, wherein the cancer is acute myeloid leukemia, bladder cancer, breast cancer, colon cancer, diffuse large B-cell lymphoma, non-small cell lung cancer, ovarian cancer, pancreatic cancer, or prostate cancer.

42. The method according to claim 39, wherein the disease or disorder associated with one or more IAPs is a pulmonary disease, disorder, or condition.

43. The method according to claim 42, wherein the pulmonary disease, disorder, or condition is chronic obstructive pulmonary disease (COPD), cystic fibrosis, or COVID-19.
